(19) United States
(12) Patent Application Publication
(10) Pub. No.: US 2026/0318914 A1
(43) Pub. Date: Oct. 15, 2026

(54) ELECTROCHEMICAL BIOSENSOR BASED ON A MXENE TRANSDUCER

(71) Applicant: King Abdullah University Of Science And Technology, Thuwal (SA)

(72) Inventors: Muhsin Ali, Thuwal (SA); Husam N. Alshareef, Thuwal (SA); Dana Zaid Alsulaiman, Thuwal (SA); Erol Hasan, Thuwal (SA)

(21) Appl. No.: 19/471,182

(22) PCT Filed: Mar. 26, 2024
(86) PCT No.: PCT/IB2024/052896

§ 371 Date: Sep. 30, 2025

Related U.S. Application Data
(60) Provisional application No. 63/454,692, filed on Mar. 26, 2023.

(51) Int. Cl. G01N 33/553; C12Q 1/00; G01N 33/543

(52) U.S. Cl. CPC G01N 33/553; C12Q 1/005; G01N 33/54353

(57) ABSTRACT

Hybrid sensing materials and electrochemical sensors incorporating the hybrid sensing materials are disclosed, as well as methods of making and methods of use thereof. The hybrid material contains a plurality of MXene sheets having biorecognition elements conjugated thereto, preferably using copper-free click chemistry. The biorecognition element contains a binding partner to an analyte of interest. The electrochemical sensor includes a working electrode, which contains a substrate having the hybrid material deposited thereon, and optionally, a counter and/or reference electrode. Electrochemical sensors incorporating the hybrid materials can be used for non-invasive detection of the absence, the presence, and/or the concentration of biomarkers, such as nucleic acids and proteins, in a biological sample, for highly sensitive (down to attomolar limit of detection), specific, and reproducible detection of target analyte in a wide linear dynamic range (from attomolar to nanomolar).

### CROSS-REFERENCE TO RELATED APPLICATION

This application claims the benefit of and priority to U.S. Provisional Application No. 63/454,692 filed Mar. 26, 2023, the content of which is incorporated herein by reference for all purpose in its entirety.

### FIELD OF THE INVENTION

This invention relates to electrochemical biosensors and methods of use thereof in detecting an analyte of interest in a sample.

### BACKGROUND OF THE INVENTION

Circulating biomarkers found in biological fluid samples (i.e. ‘liquid biopsies’) including cell-free nucleic acids (cfNAs) have been reported as promising diagnostic, prognostic and monitoring biomarkers for a broad range of diseases, including cancer, neurodegenerative diseases and stroke [Rainer, et al. Ann. N. Y. Acad. Sci. 2006, 1075, (1), 271-277; Swarup, et al. FEBS Lett. 2007, 581, (5), 795-799; Danese, et al., Semin. Thromb. Hemost. 2014, 40, (07), 766-773; Fleischhacker, et al., BBA-Rev Cancer 2007, 1775, (1), 181-232.]. cfNAs are DNA and RNA molecules found freely circulating in body fluids such as urine, blood, and saliva. While small amounts of cfNAs (10 s of ng mL-1) are physiologically released by cells during apoptosis and necrosis in healthy individuals, research has revealed that abnormal levels of these molecules are present in individuals with clinical disorders including cancer, neurodegenerative diseases, autoimmune disorders and stroke. Consequently, the ability to accurately and efficiently detect circulating biomarkers in biological fluids has the potential to offer a promising and minimally-invasive approach for the diagnosis and management of a range of acute pathologies. Among the most promising cfNAs is microRNA which represents a class of short (22-25 nt) non-coding RNA, with great promise as clinical biomarkers due to their gene regulatory roles and dysregulated patterns in many age-associated diseases including cancer, neurodegenerative diseases, and cardiovascular diseases. While highly promising biomarkers, their short lengths, low concentrations, and high sequence homology make them challenging to detect even with gold-standard methods, which are complex, semiquantitative, time-intensive, and costly. One promising approach for the detection of rare and clinically-relevant circulating biomarkers development of advanced microRNA biosensors relies on electrochemical strategies.

Many approaches have been reported in the literature for electrochemical detection of biomarkers like microRNA. However, they often rely on the use of expensive enzymes for target or signal amplification and/or the use of labelled small-molecule or nanomaterial-based probes. Such strategies lead to expensive, arduous, and time-consuming techniques that are not amenable for point-of-care devices and often suffer poor reproducibility.

There is therefore a need for improved biomarker sensors such as microRNA sensors that are stable, simple, and cost-effective, and yet sensitive, selective, and reproducible.

It is an object of the present invention to provide materials for use in electrochemical sensors.

It is another object of the present invention to provide electrochemical sensors for detection of analyte in a sample.

It is also an object of the present invention to provide methods for making the electrochemical sensors for detection of analyte in a sample.

It is still an object of the present invention to provide methods for detecting an analyte of interest in a biological sample, using the electrochemical sensors.

### SUMMARY OF THE INVENTION

Hybrid sensing materials (also referred to herein as “hybrid materials”) and electrochemical sensors (also referred to herein as “sensors”) incorporating the hybrid sensing materials are disclosed herein, as well as methods of making and methods of use thereof. The hybrid material contains a plurality of MXene sheets having biorecognition elements conjugated thereto. The biorecognition element contains a binding partner to an analyte of interest (also referred to herein as “target analyte”). In some forms, the biorecognition element is a protein or peptide nucleic acid (PNA) which is specifically designed. The disclosed hybrid material incorporating a MXene-biorecognition element hybrid material, allows for amplification-free, enzyme-free, and target-specific detection of a variety of analytes, such as nucleic acids (e.g., miRNA, viral RNA), proteins, extracellular vesicles, cells, pathogens, and components of pathogens.

Typically, the recognition elements are conjugated to one or more surfaces of the MXene sheets via a linker. The linker is typically formed by an alkoxysilane or a phosphonic acid; and a moiety produced by Click Chemistry. In some forms, the linker can be represented by the formula:

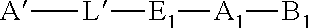

wherein A′ can be an alkoxysilane or phosphonic acid; L′ can be

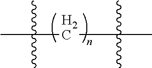

or

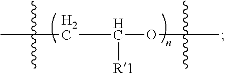

each occurrence of n can be independently an integer from 1 to 20, for example 1, 2, 2, 4, 5, 6, 7, 8, 9, 10, 11, 12, 13, 14, 15, 16, 17, 18, 19, 20; R′₁ can be hydrogen or C₁-C₄ unsubstituted alkyl (e.g., methyl, ethyl, etc.); E₁ can be a bond, a peptide, or

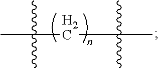

A₁ can be a moiety formed by click chemistry; and B₁ can be the biorecognition element.

In some forms, A′ can be

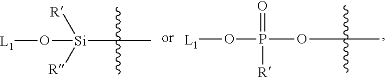

wherein L₁ can be an attachment point to the surface of MXene; R′ and R″ can be independently

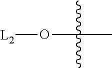

or a C₁-C₄ unsubstituted alkyl; and L₂ can be an attachment point to the surface of MXene, a C₁-C₄ unsubstituted alkyl, or hydrogen.

In some forms, the linker can be represented by the formula:

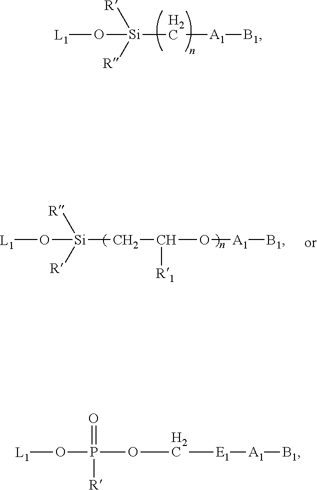

wherein L₁, R′, R″, R′₁, n, A₁, B₁, and E₁ can be as defined above.

In some forms, A₁ can be

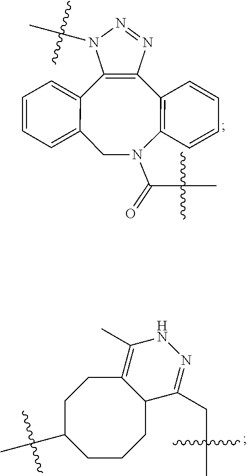 [SMILES: *C(=O)N1Cc2ccccc2-c2c(nnn2*)-c2ccccc21 ; *CC1=NNC(C)=C2CCC(*)CCCC12]

or

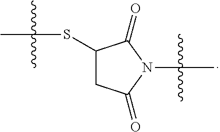 [SMILES: *SC1CC(=O)N(*)C1=O]

In some forms, A₁ can be

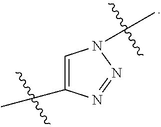 [SMILES: *c1cn(*)nn1]

In some forms, one of R′ and R″ is

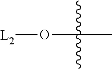

and L₂ is an attachment point to the one or more surfaces of the MXene sheets. In some forms, both R′ and R″ are

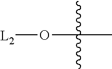

and L₂ is an attachment point to the one or more surfaces of the MXene sheets.

Sensors incorporating the hybrid material disclosed herein are stable, measured by X-ray photoelectron spectroscopy (XPS), and highly sensitive (down to attomolar limit of detection) with a wide linear dynamic range (from attomolar to nanomolar) and high target specificity towards the target analyte. Performance or signal stability was demonstrated by repeated CV measurements in standard redox label (ferrocyanide) showing overlapping CV curves and thus no loss of MXene or degradation of sensor over repeat measurements. Repeatability was demonstrated by establishing that the same electrode can be used for multiple measurements without significant loss of signal (i.e., signal change ≤20%). The sensor includes a working electrode, which contains a substrate and the hybrid material. The substrate may be electrically conductive or non-conductive. The hybrid material is deposited on one or more surfaces of the substrate. The sensor may further contain one or more additional electrodes, for example, a counter electrode and optionally a reference electrode.

Also disclosed are methods of making the hybrid materials. The method includes: (a) treating MXene sheets having a plurality of surface hydroxyl groups with a clickable group functionalized silane or phosphonic acid having the structure of

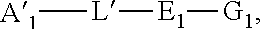

wherein A′₁ can be

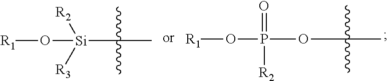

R₁ can be a C₁-C₄ unsubstituted alkyl; R₂ and R₃ can be independently

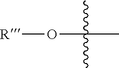

or a C₁-C₄ unsubstituted alkyl; R′″ can be hydrogen or a C₁-C₄ unsubstituted alkyl; L′ can be

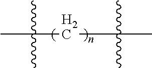 [SMILES: *C*]

or

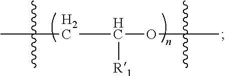

each occurrence of n can be independently an integer from 1 to 20, for example 1, 2, 2, 4, 5, 6, 7, 8, 9, 10, 11, 12, 13, 14, 15, 16, 17, 18, 19, 20; R′₁ can be hydrogen or C₁-C₄ unsubstituted alkyl (e.g., methyl, ethyl, etc.); E₁ can be a bond, a peptide, or

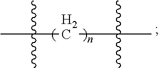 [SMILES: *C*]

and G₁ can be a clickable group, to form clickable group functionalized MXene sheets, and (b) reacting the clickable group functionalized MXene sheets with a clickable group functionalized recognition element G₂-B₁, wherein B₁ is the recognition element and G₂ is a corresponding clickable group, to form the hybrid material.

In some forms, the clickable group functionalized silane or phosphonic acid in step (a) can have the structure of

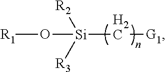

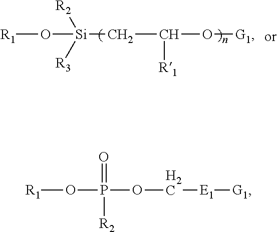

weren R₁, R₂, R₃, R′₁, n, E₁, and G₁ can be as defined above.

The corresponding clickable group G₂ of the clickable group functionalized recognition element in step (b) can be any suitable functional group capable of reacting with the clickable group G₁ on the MXene via Click Chemistry.

For example, G₁ can be

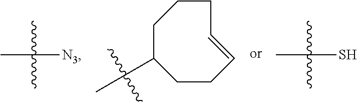

### and G₂ can be

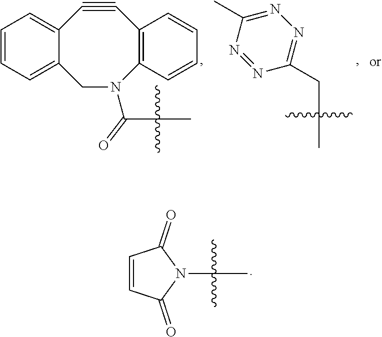

### Alternatively, G₁ can be

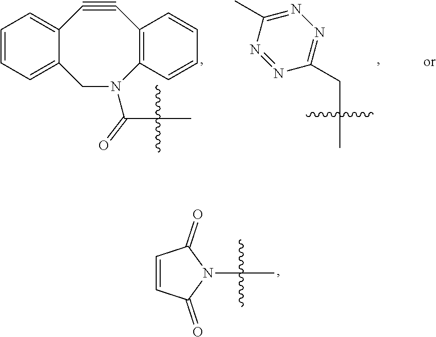

### and G₂ can be

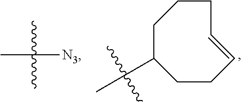

or

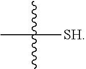 [SMILES: *S]

For example, G₁ can be

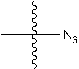 [SMILES: *N=[N+]=[N-]]

### and G₂ can be

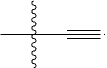 [SMILES: *C#C]

### Alternatively, G₁ can be

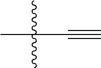 [SMILES: *C#C]

### and G₂ can be

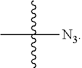 [SMILES: *N=[N+]=[N-]]

Methods for detecting an analyte of interest in a biological sample using the sensors are also disclosed. The method includes: (i) contacting the working electrode of the sensor with the biological sample; (ii) optionally contacting the working electrode of the sensor with a redox reporter solution; and (iii) measuring an electrochemical signal generated by the sensor. In some forms, when step (ii) is included in the method, steps (i) and (ii) can be performed simultaneously. Optionally, the method further includes: (iv) contacting the working electrode of the sensor with the redox reporter solution; and (v) measuring a background signal generated by the sensor, wherein steps (iv) and (v) are performed prior to step (i). The electrochemical signal and background signal of the sensor may be measured using any suitable electrochemical methods, such as cyclic voltammetry, differential pulse voltammetry, square wave voltammetry, or amperometry, or a combination thereof.

Generally, an electrochemical signal that is more intense compared to the background signal is indicative of the presence of the analyte of interest in the biological sample; an electrochemical signal that is similar to the background signal (or less than three times the standard deviation of the control, background, blank or non-target signal) is indicative of the absence of the analyte of interest in the biological sample; and different concentrations of the analyte of interest in the biological sample can be determined based on the intensity level of the electrochemical signal compared to the background signal. To be quantitative, any signal less than three times the standard deviation of the background signal is indicative of the absence of target analyte

The methods disclosed herein allow highly sensitive (down to attomolar limit of detection), specific, and reproducible detection of target analyte over a wide linear dynamic range (from attomolar to nanomolar). Without being bound by theory, the outstanding analytical performance of the sensors disclosed herein may be attributed to the distinct electrical conductivity, morphology, and surface functional groups of the 2D ultrathin MXene sheets in combination with the highly efficient and stable click chemistry used for functionalization, resulting in a surprisingly active nanohybrid transducer surface of the working electrode offering enhanced bioreceptor loading and rapid target biomarker access.

### BRIEF DESCRIPTION OF THE DRAWINGS

FIG. 1A is a schematic showing the process of functionalizing MXene to introduce azide groups. FIG. 1B is a schematic showing the process of preparing and using the working electrode. For preparing the working electrode (PNA-Ti₃C₂Tx-MXene-GCE), GCE was modified with azide functionalized MXene and DBCO-PNA was immobilized to the MXene via click reaction to form PNA/Ti₃C₂Tx-MXene nanohybrid. To function as a biosensor, the nanohybrid is deposited on the working electrode, then target analyte (for example miR-141) was introduced to the surface of the working electrode, followed by incubation with redox probe (MB).

FIG. 2A is a graph showing the XPS spectra of Ti₃C₂Tx-MXene, AzPTES-Ti₃C₂Tx-MXene, and BP Fluor 647 DBCO MXene. The BP Fluor 647 DBCO is used as a model or proxy for a bioreceptor functionalized with DBCO. FIG. 2B is a graph showing the highlighted peaks in FIG. 2A. FIG. 2C is a graph showing the Si peak in FIG. 2A. FIG. 2D is a graph showing the high resolution XPS spectrum of azido peaks of AzPTES-Ti₃C₂Tx. FIG. 2E is a graph showing the S peak in FIG. 2A. FIG. 2F is a graph showing the zeta potential of Ti₃C₂Tx before and after azido functionalization. FIG. 2G is a graph showing the DLS spectrum of pure MXene nanosheets.

FIGS. 3A-3C are EDX mapping of pure Ti₃C₂Tₓ MXene (FIG. 3A), azido-functionalized Ti₃C₂Tₓ MXene (FIG. 3B), and Ti₃C₂Tₓ MXene post-click reaction (FIG. 3C). The additional elements (Si, N, S) followed by each step confirms the successful modification and immobilization of the PNA via Cu-free click reaction. In particular, the appearance of the Si and N peaks indicate successful silanization. The appearance of the S peaks indicates successful click reaction with the model bioreceptor (BP Fluor 647 DBCO which contains S atoms). FIG. 3D is a graph showing Raman spectra of the bare, and functionalized (post-silanization) Ti₃C₂Tₓ MXene.

FIG. 4A is a graph showing cyclic voltammograms recorded at bare GCE and MXene-GCE in 5 mM [Ru (NH₃)₆]³⁺, respectively, by scanning the potential from −0.6 to 0.2 V at a scan rate of 100 mVsec⁻¹. FIG. 4B is a graph showing the cyclic voltammogram recorded at bare GCE of FIG. 4A. FIG. 4C is a graph showing cyclic voltammograms recorded at PNA-Ti₃C₂ MXene-GCE at different scan rates from 20 to 140 mV/s. FIG. 4D is a graph showing the plots of anodic and cathodic currents versus square root of scan rate. FIG. 4E is a graph showing the electrochemical impedance spectroscopy of bare GCE, Ti₃C₂ MXene coated GCE, PNA-functionalized Ti₃C₂ MXene in the absence of target miRNA, and PNA-functionalized Ti₃C₂ MXene after hybridization with the target miRNA. The circuit diagram is shown in the inset of FIG. 4E. FIG. 4F is a graph showing the kinetics mechanism of the f-Ti₃C₂-MXene/GC electrode at scan rates ranging from 20 to 120 mVs⁻¹. FIG. 4G is a graph showing the increase of cathodic and anodic current with the square root of the scan rate. FIG. 4H is a graph showing the cyclic voltammograms of MXene coated GCE almost under multiple scans.

FIG. 5A is a graph showing cyclic voltammograms of the working electrode in the presence or absence of target miRNA and after MB incubation, at a scan rate of 100 mV/sec. FIG. 5B is a schematic diagram showing the redox label reaction through intercalator dye (MB) in the absence (upper panel) or presence (lower panel) of target miRNA. FIG. 5C is a graph showing cyclic voltammograms in the presence of target microRNA in concentrations ranging from 1 fM to 1 nM. FIG. 5D is a graph showing the oxidation peaks of the cyclic voltammograms recorded at various microRNA concentrations in FIG. 5C. FIG. 5E is a graph showing the calibration curve of the current response of PNA-Ti₃C₂-MXene-GCE at different concentrations of miRNA. FIG. 5F is a graph showing the differential pulse voltammetry (“DPV”) response of the PNA-Ti₃C₂-MXene-GCE at different miRNA concentrations in 1×TE buffer. FIG. 5G is a graph showing the calibration curve of DPV peak currents at target miRNA concentrations ranging from 1 fM to 1 nM. FIG. 5H is a graph showing the calibration curve for the detection of miRNA-141 based on two different sequences of PNA (7 and 17 mers). FIG. 5I is a schematic diagram showing the MB intercalation using two different sequences of PNA (7 and 17 mers).

FIG. 6 shows specificity data obtained using non-target microRNA.

FIG. 7 shows the effect of time and storage conditions on biosensor stability. Sensor performance was evaluated by quantifying the oxidation peak current of four sensors in the presence of 10 mM RuHex probe. Sensors 1 and 2 were tested on the fabrication day (i.e., fresh Sensors), with the signal of Sensor 1 normalized to 100%. Sensors 3 and 4 were examined after a 7-day storage at 4° C.

### DETAILED DESCRIPTION OF THE INVENTION

### I. Definitions

The term “conditions sufficient for” refers to any environment that permits the desired activity, for example, that permits specific binding or hybridization between two nucleic acid molecules or that permits reverse transcription and/or amplification of a nucleic acid. Such an environment may include, but is not limited to, particular incubation conditions (such as time and/or temperature) or presence and/or concentration of particular factors, for example in a solution (such as buffer(s), salt(s), metal ion(s), detergent(s), nucleotide(s), enzyme(s), etc.).

As used herein, the terms “nucleic acid”, “polynucleotide” and “oligonucleotide” refer to primers, probes, oligomer fragments, and oligomer controls and are generic to polydeoxyribonucleotides (containing 2-deoxy-D-ribose), to polyribonucleotides (containing D-ribose), and to any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA.

As used herein, the terms “detect” or “detecting generally refer to obtaining information. Detecting or determining can utilize any of a variety of techniques available to those skilled in the art, including for example specific techniques explicitly referred to herein. Detecting may involve manipulation of a physical sample, consideration and/or manipulation of data or information, for example utilizing a computer or other processing unit adapted to perform a relevant analysis, and/or receiving relevant information and/or materials from a source. Detecting may also mean comparing an obtained value to a known value, such as a known test value, a known control value, or a threshold value. Detecting may also mean forming a conclusion based on the difference between the obtained value and the known value.

The terms “contact”, “contacting” or “bringing into contact” describe placement in physical association for example, in solid and/or liquid form. For example, contacting or combining can occur in vitro with one or more primers and/or probes and a biological sample (such as a sample including nucleic acids) in solution.

“Amplification” refers to increasing the number of copies of a nucleic acid molecule, such as a gene, fragment of a gene, or other genomic region. The products of an amplification reaction are called amplification products or amplicons.

The terms “complement”, “complementary” or “complementarity” as used herein with reference to polynucleotides (i.e., a sequence of nucleotides such as an oligonucleotide or a target nucleic acid) refer to the Watson/Crick base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5′.” Certain bases not commonly found in naturally occurring nucleic acids may be included in the nucleic acids described herein. These include, for example, inosine, 7-deazaguanine, Locked Nucleic Acids (LNA), and Peptide Nucleic Acids (PNA). Complementarity need not be perfect (e.g., it can be partial or complete); stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement The terms “target nucleic acid” or “target sequence” or “target segment” as used herein refer to a nucleic acid sequence of interest to be detected and/or quantified in the sample to be analyzed. Target nucleic acid may be composed of segments of a genome, a complete gene with or without intergenic sequence, segments or portions of a gene with or without intergenic sequence, or sequence of nucleic acids to which probes or primers are designed to hybridize. Target nucleic acids may include a wild-type sequence(s), a mutation, deletion, insertion or duplication, tandem repeat elements, a gene of interest, a region of a gene of interest or any upstream or downstream region thereof. Target nucleic acids may represent alternative sequences or alleles of a particular gene. Target nucleic acids may be derived from genomic DNA, cDNA, or RNA. sequence, and can also be a cDNA.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

### II. Compositions

Hybrid sensing materials (also referred to herein as “hybrid materials”) and electrochemical sensors (also referred to herein as “sensors”) incorporating the hybrid sensing materials are provided herein, as well as methods of making and methods of use thereof.

The hybrid material contains a plurality of MXene sheets having biorecognition elements conjugated thereto. The biorecognition element contains a binding partner to an analyte of interest (also referred to herein as “target analyte”), such as a bespoke PNA. The disclosed hybrid material is designed to provide a stable and highly active electrochemical transducer surface when incorporated on a surface of a substrate. Without being bound by theory the high electrochemical productivity of the hybrid material may be attributed to a high loading of the biorecognition element on the MXene sheets (i.e., at least 1 moles based on the amount of the MXene) and rapid access of target analytes to the biorecognition elements due to the porous nature of the hybrid material. For example, molar amount of PNA functionalized on MXene can be obtained by quantifying new azido peaks or nitrogen peaks from XPS. For example, in 3-azidopropyltriethoxysilane composition, the mole ratio between Si and N₃-functionality is 1:1. Hence, Si wt % can be used for the calculation of N₃-functionality moles in the N₃-modified MXene. 6.78 g of Si in 100 g of N₃-modified MXene corresponds to 0.242 moles of Si or N₃-functionality. Hence, 1 mg of N₃-modified MXene has 2.42 μmols of N₃-functionality.

The sensor includes a working electrode, which contains a substrate and the hybrid material. The substrate may be electrically conductive or non-conductive. The hybrid material is deposited on one or more surfaces of the substrate. The sensor may further contain one or more additional electrodes, for example, a counter electrode and optionally a reference electrode. Sensors incorporating the disclosed hybrid material are stable and can be used for detection of an analyte of interest in a biological sample or an extract therefrom with high sensitivity (down to attomolar limit of detection), high reproducibility, a wide linear dynamic range (from attomolar to nanomolar), and high target specificity towards the target analyte (with single-nucleotide resolution).

It is believed that the disclosed hybrid material is the first demonstration of a MXene-biorecognition element hybrid material, which, when used in an electrochemical sensor, allows amplification-free, enzyme-free, and target-specific detection of a variety of analytes, such as nucleic acids (e.g., miRNA, mRNA, ssDNA), proteins, pathogens, components of pathogens, hormones, carbohydrates, cells, extracellular vesicles, and viruses. Without being bound to any theories, the outstanding analytical performance of the sensors disclosed herein may be attributed to the distinct electrical conductivity, morphology, and surface functional groups of the 2D ultrathin MXene sheets in combination with the highly efficient and stable click chemistry used for functionalization with biorecognition element, resulting a surprisingly active nanohybrid transducer surface for binding with the target analytes and electron transfer.

### A. Hybrid Sensing Material

The hybrid material contains a plurality of MXene sheets having biorecognition elements conjugated thereto. This conjugation preferably involves a chemical or covalent bond rather than most other functionalization strategies with are based on weaker non-covalent interactions. The biorecognition element contains a binding partner to an analyte of interest (also referred to herein as “target analyte”). Preferably, the biorecognition elements are conjugated to the MXene sheets via a linker formed by click chemistry, providing a stable sensing material with a high loading of the biorecognition elements for binding to target analytes and a porous structure for rapid access of the target analytes to the biorecognition elements.

### 1. MXene Sheets

The MXene of the hybrid material are 2D transition metal carbide, metal nitride, or metal carbonitride materials having large surface area, high metallic conductivity, and good biocompatibility. In particular, the high conductivity of MXene allows it to act as charge-transfer promoters and as catalysts to facilitate electrochemical reactions.

The MXene used to form the hybrid material is typically in the form of a sheet. Typically, the MXene sheet has a layered body containing two or more layers. The layered body of MXene sheet can be represented by Mₙ₊₁Xₙ, wherein n can be an integer from 1 to 4, each M can be a metal of Group 3, 4, 5, 6, or 7, and each X can be a carbon atom or a nitrogen atom. In some forms, each M of the layered body can be Ti, Zr, Hf, V, Nb, Ta, Cr, or Mo. In some forms, the MXene sheet includes repeating units in which each layer of X atoms is disposed in between adjacent layers of n+1 layers of M atoms. Preferably, each M layer of the layered body is Ti. In preferred forms, the layered body of MXene sheet is Ti₃C₂. Additional examples of MXene sheets have a layered body represented by: Sc₂C, Ti₂C, Ti₂N, Zr₂C, Zr₂N, Hf₂C, Hf₂N, V₂C, V₂N, Nb₂C, Ta₂C, Cr₂C, Cr₂N, Mo₂C, Mo_{1.3}C, Cr_{1.3}C, (Ti,V)₂C, (Ti,Nb)₂C, W₂C, W_{1.3}C, Mo₂N, Nb_{1.3}C, Mo_{1.3}Y_{0.6}C (in the above formulae, “1.3” and “0.6” mean about 1.3 (=4/3) and about 0.6 (=2/3), respectively), Ti₃N₂, Ti₃(CN), Zr₃C₂, (Ti,V)₃C₂, (Ti₂Nb)C₂, (Ti₂Ta)C₂, (Ti₂Mn)C₂, Hf₃C₂, (Hf₂V)C₂, (Hf₂Mn)C₂, (V₂Ti)C₂, (Cr₂Ti)C₂, (Cr₂V)C₂, (Cr₂Nb)C₂, (Cr₂Ta)C₂, (Mo₂Sc)C₂, (Mo₂Ti)C₂, (Mo₂Zr)C₂, (Mo₂Hf)C₂, (Mo₂V)C₂, (Mo₂Nb)C₂, (Mo₂Ta)C₂, (W₂Ti)C₂, (W₂Zr)C₂, (W₂Hf)C₂, Ti₄N3, V₄C₃, Nb₄C₃, Ta₄C₃, (Ti,Nb)₄C₃, (Nb,Zr)₄C₃, (Ti₂Nb₂)C₃, (Ti₂Ta₂)C₃, (V₂Ti₂)C₃, (V₂Nb₂)C₃, (V₂Ta₂)C₃, (Nb₂Ta₂)C₃, (Cr₂Ti₂)C₃, (Cr₂V₂)C₃, (Cr₂Nb₂)C₃, (Cr₂Ta₂)C₃, (Mo₂Ti₂)C₃, (Mo₂Zr₂)C₃, (Mo₂Hf₂)C₃, (Mo₂V₂)C₃, (Mo₂Nb₂)C₃, (Mo₂Ta₂)C₃, (W₂Ti₂)C₃, (W₂Zr₂)C₃, and (W₂Hf₂)C₃.

Optionally, the layered body of MXene sheet contains one or more surface functional groups, on one or more surfaces of the layered body, prior to and optionally after conjugation with the recognition elements. For example, the layered body of MXene sheet contains a plurality of surface functional groups on a top surface, a bottom surface, and one or more side surface(s), prior to and optionally after conjugation with the recognition elements. When one or more surface functional groups are present on the MXene sheet, the layered body can be represented by Mₙ₊₁XₙTₚ, wherein T refers to the surface functional group and p can be any number. The surface functional groups on the surface(s) of the layered body of MXene sheet can be any suitable organic moieties, such as hydroxyl, oxide, halogen (e.g., —F, —Br, —Cl, and —I), or carbonyl (—C(═O)—R′, wherein R′ can be an unsubstituted C₁-C₆ alkyl or OH), or a combination thereof. Preferably, the layered body of MXene sheet contains a plurality of hydroxyl groups on one or more surfaces of the layered body. For example, the layered body of MXene is represented by Ti₃C₂Tp, wherein T is hydroxyl and p can be any number.

In some forms, the layered body of MXene sheet has a high density of surface functional groups, prior to conjugation with the biorecognition elements. In these forms, at least 50 mol %, at least 60 mol %, at least 70 mol %, at least 80 mol %, or at least 90 mol % of the surface functional groups are conjugated to biorecognition elements. For example, prior to conjugation with the biorecognition elements, the layered body of MXene sheet has a high density of surface hydroxyl groups on one or more surfaces of the layered body; after conjugation, at least 90 mol % of the hydroxyl groups are conjugated to the biorecognition elements.

The MXene sheet can be synthesized using methods known in the art, for example, using selectively etching (removing and optionally layer-separating) A atoms (and optionally parts of M atoms) from a MAX phase represented by the formula: Mₙ₊₁AXₙ, wherein M, X, and n are as described above and A is at least one element of Group 12, 13, 14, 15, or 16, normally an element of Group A, typically of Group IIIA and Group IVA, such as Al, Ga, In, Tl, Si, Ge, Sn, Pb, P, As, S, and Cd, and preferably Al. The MAX phase can have a crystal structure in which a layer composed of A atoms is located between the two layers of Mₙ₊₁Xₙ (i.e., can have a crystal lattice in which each X is located in the octahedral array of M). The MAX phase can include repeating units in which each layer of X atoms is disposed in between adjacent layers of the n+1 layers of M atoms, and a layer of A atoms (“A atom layer”) is disposed as a layer next to the (n+1)th layer of M atoms. The A atom layer (and optionally a part of the M atoms) is removed by selectively etching (removing and optionally layer-separating) the A atoms (and optionally a part of the M atoms) from the MAX phase. In some forms, by selectively etching the A atoms (and optionally a part of the M atoms) from the MAX phase, the A atom layer (and optionally a part of the M atoms) is removed, and functional groups (e.g., hydroxyl, oxide, halogen, carbonyl, etc.) existing in the etching solution (such as an aqueous solution containing acid) are modified on the exposed surface of the Mₙ₊₁Xₙ layered body. In some forms, the MXene sheet may contain remaining A atoms at a relatively small amount, for example, 10% by weight or less, 8% by weight or less, or 6% by weight or less relative to the original amount of A atoms.

MXene is a 2D nanomaterial with lateral (width and height) dimensions in the microscale but thickness (per sheet) in the nanoscale. The working electrode is coated with many of these individual 2D nanomaterials forming a layered coating that is highly porous and having a high surface area to volume ratio. The dimensions of MXene sheet can be determined using methods known in the art, such as by using images taken by a scanning electron microscope (SEM), a transmission electron microscope (TEM), or an atomic force microscope (AFM), or by calculation from the positions on the reciprocal lattice space of the (002) plane measured by an X-ray diffraction (XRD) method.

### 2. Biorecognition Elements

The biorecognition elements are conjugated to one or more surfaces of the MXene sheets. The conjugation is typically formed by reacting a clickable functional group on the biorecognition element with a corresponding clickable functional group on the MXene sheet. Therefore, the biorecognition element is typically conjugated to the surface(s) of the MXene sheet via a chemical linker formed by click chemistry. The biorecognition element contains a binding partner to an analyte of interest (also referred to herein as “target analyte”). The biorecognition element is oriented on the surface(s) of the MXene sheet such that the binding partner is exposed for interaction with target analytes in a biological sample with which it is contacted. Optionally, the biorecognition element further contains a label, such as a dye molecule, to aid characterization of the hybrid material.

Exemplary biorecognition elements for use in the hybrid material include, but are not limited to, nucleic acids, peptides, proteins (e.g., antibodies, nanobodies, antibody fragments, enzymes, etc.). For example, the biorecognition element for use in the hybrid material is a peptide nucleic acid (“PNA”), which is or contains a sequence that is complementary to a target nucleic acid (such as miRNA) in a biological sample, such that the PNA can bind to the target nucleic acid when the hybrid material and the biological sample are brought in contact with each other.

a. Peptide Nucleic Acids

PNAs are synthetic pseudo-peptide oligomers, in which the negatively-charged sugar-phosphate backbone of natural nucleic acids is replaced with a polyamide backbone. Owing largely to their neutral backbone, PNAs have higher chemical and thermal stability as well as binding affinity to target nucleic acids, even at low ionic strength conditions. Further, PNAs suitable for use to form the hybrid materials contain at least a sequence that is complementary to a target nucleic acid in a biological sample, which allows specific interaction with the target nucleic acid and thus specific detection of the target nucleic acid. PNAs hybridize to complementary DNA or RNA in a sequence-dependent manner, according to the Watson-Crick hydrogen bonding scheme. The high specificity and neutral charge of PNA, in combination with the electrochemical property of MXene sheets and high loading of PNA on the MXene, allows ultrasensitive and specific detection of nucleic acids, such as microRNA.

For example, PNA that is or contains a sequence complementary to a target nucleic acid (such as a miRNA) is covalently attached onto one or more surfaces of Ti₃C₂ MXene sheets using bio-orthogonal copper-free click chemistry, which can achieve highly stable and covalent immobilization and high loading density of PNA at room temperature. The formed PNA-Ti3C2 MXene hybrid material can be used for hybridization and detection of the target nucleic acids, such as microRNA sequence.

The PNA that is or contains a sequence complementary to a target nucleic acid (such as miRNA) can have any suitable length, such as ranging from 5 mer to 20 mer or longer sequences, preferably from 7 mer to 17 mer, such as 17 mer. In some forms, the length of the PNA affects the electrochemical signal obtained upon binding to a target nucleic acid. For example, when used in a sensor, a 17 mer PNA produces a higher electrochemical signal compared to a 7 mer PNA, upon binding with a target nucleic acid.

The PNAs containing a target nucleic acid-specific sequence can be conveniently designed based on the sequence of specific target nucleic acids, and may be commercially available or synthesized using methods known in the art, see, for example, https://shop.biosearchtech.com/support/nac/peptide-nucleic-acid-pna-synthesis. Specific exemplary PNAs are provided in Table 1 in the Examples below.

The PNA is complementary to the target nucleic acid.

Exemplary miRNAs include miR-122, miR-192, miR-21, miR-223, miR-375, miR-30a, miR-33a, miR-34a, miR-16, miR-155, miR-132, miR-27a, miR-150, miR-199, miR-200, miR-17, miR-214, miR-9, miR-29a, miR-212, miR-214, miR-497, miR-378, miR-320, miR-222, miR-106a, miR-92, miR-20, miR-23, miR-18, miR-126, miR-141 and Cel-miR-39.

### 3. Linker

Typically, the biological elements are conjugated to the surface(s) of MXene by reacting a functional group on the biorecognition element with a corresponding functional group on the MXene sheet, which in turn forms a chemical linker (also referred to herein as “linker”). The functional group and corresponding functional group on the biorecognition element and MXene sheet refer to two chemical groups between which a conjugation reaction can occur under suitable reaction conditions. Preferably, the reaction occurs between the functional group of biorecognition elements and the corresponding functional group of the MXene is click chemistry. In these forms, the biorecognition elements are conjugated to the MXene sheets via a linker formed by click chemistry, providing a stable sensing material with a high loading of the biorecognition elements for binding to target analytes and a porous structure for rapid access of the target analytes to the biorecognition elements. Preferably, the linker contains an alkoxysilane or phosphonic acid and a moiety formed by Click Chemistry.

In some forms, the linker formed by click chemistry can have the structure of:

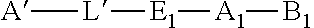

wherein A′ can be an alkoxysilane or phosphonic acid; L′ can be or

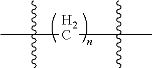

or

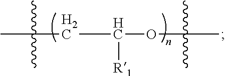

each occurrence of n can be independently an integer from 1 to 20, for example 1, 2, 2, 4, 5, 6, 7, 8, 9, 10, 11, 12, 13, 14, 15, 16, 17, 18, 19, 20; R′₁ can be hydrogen or C₁-C₄ unsubstituted alkyl (e.g., methyl, ethyl, etc.); E₁ can be a bond, a peptide, or

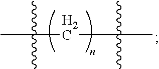

A₁ can be a moiety formed by click chemistry; and B₁ can be the biorecognition element, such as any one of those described above, for example a PNA.

In some forms, A′ can be

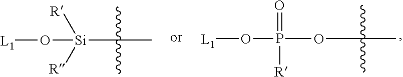

wherein L₁ can be an attachment point to the surface of MXene; R′ and R″ can be independently

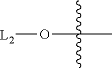

or a C₁-C₄ unsubstituted alkyl; and L₂ can be an attachment point to the surface of MXene, a C₁-C₄ unsubstituted alkyl, or hydrogen.

In some forms, the linker can be represented by the formula:

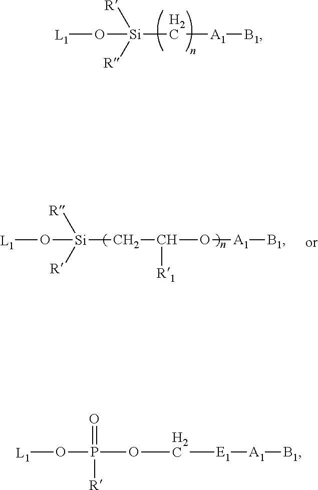

wherein L₁, R′, R″, R′₁, n, A₁, B₁, and E₁ can be as defined above.

In some forms, A₁ can be

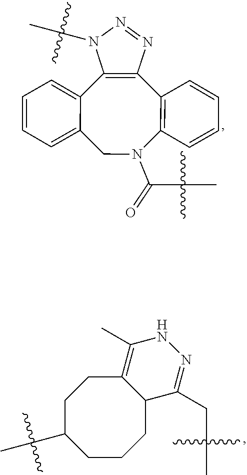 [SMILES: *C(=O)N1Cc2ccccc2-c2c(nnn2*)-c2ccccc21 ; *CC1=NNC(C)=C2CCC(*)CCCC12]

or

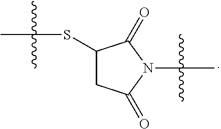 [SMILES: *SC1CC(=O)N(*)C1=O]

In some forms, A₁ can be

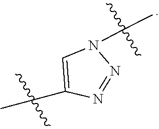 [SMILES: *c1cn(*)nn1]

In some forms, one of R′ and R″ is

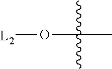

and L₂ can be an attachment point to the one or more surfaces of the MXene sheets. In some forms, both R′ and R″ are

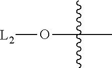

and L₂ can be an attachment point to the one or more surfaces of the MXene sheets.

### B. Electrochemical Sensor

The disclosed electrochemical sensor includes a working electrode, which contains a substrate and a hybrid material described above. The substrate may be electrically conductive or non-conductive. Optionally, the sensor further includes a counter electrode and/or a reference electrode. For example, the sensor further includes a counter electrode. The hybrid material is deposited on a surface of the substrate, optionally more than one surface of the substrate, and forms a coating on the surface of the substrate. In some forms, the coating formed by the hybrid material on the surface of the substrate is porous, thereby provides easy access for target analytes to diffuse in and interact with the biorecognition elements of the hybrid material.

In some forms, the disclosed sensor does not include any enzymes and does not include any nanoparticles, which are typically needed in electrochemical sensors previously reported in the art, in particular for miRNA detection.

Electrochemical sensors incorporating the hybrid material disclosed herein are stable (e.g., the sensor has a shelf stability of at least 1 week, at least 1 month, at least 3 months, or at least 6 months, at about 4° C.) and highly sensitive (down to attomolar limit of detection) with a wide linear dynamic range (from attomolar to nanomolar) and high target specificity towards the target analyte.

Further, the sensors can be easily integrated on a support for point-of-care testing. For example, the working electrode and optionally the counter and/or reference electrode are integrated on a microfluidic chip for point-of-care testing of target analytes in a biological sample, such as miRNA in blood or saliva.

Electrochemical sensors incorporating the hybrid material as disclosed herein demonstrate excellent specificity. Exemplary embodiments showed using microRNA demonstrate that even the microRNA with only a single mutation difference from the target (i.e. miR-141-1SNP) generated below 30% signal, indicating the extremely high sequence specificity of the sensor using PNA probes as selective bioreceptors.

### 1. Working Electrode

The working electrode of the electrochemical sensor disclosed herein contains a substrate and a hybrid material described above. The substrate may be electrically conductive or non-conductive. The hybrid material is deposited on a surface of the substrate using a suitable method, such as by drop-casting or spin-coating. When deposited on the surface of the substrate, the hybrid material of the working electrode can form a coating on the surface of the substrate. In some forms, the coating formed on the surface of the substrate is porous, providing easy access for target analytes to diffuse in and interact with the biorecognition elements of the hybrid material. Further, the coating formed on the surface of the substrate is stable. For example, the working electrode has a shelf stability of at least 1 week, at least 1 month, at least 3 months, or at least 6 months, at room temperature or in a refrigerator (i.e., at about 4° C.). Optionally, the working electrode has a shelf stability of at least 1 week, at least 1 month, at least 3 months, or at least 6 months, at about 4° C., under conditions that prevent oxidation of the MXene, such as under an inert atmosphere. Additional storage conditions that can reduce/prevent oxidation of MXene are described in Iqbal, et al., Nano Convergence, 8:9 (2021).

The deposition of the functionalized-MXene on the working electrode affects the long-term stability of the sensor when incubated in a solution. It is important to ensure that the MXene does not flake off or come off the electrode surface over time and as a result of applying current or voltage to the electrode. In some forms, the methods achieve a strong MXene-electrode binding by adding Nafion (polymer) into the mixture to help create a stable layer (as adhesive) on the electrode. Conductive stability tests by cyclic voltammetry measurements of the assembled MXene-electrode in a standard redox solution which shows overlapping curves over time can be used to demonstrate stability of the coating or strength of the coating.

a. Substrate

The substrate of the working electrode may be formed from an electrically conductive or non-conductive material.

The substrate of the working electrode, electrically conductive or non-conductive, has a cross-section of any shape appropriate, such as a circle, a square, a rectangular, an ellipse, a parabola, a hyperbola, etc. In some forms, the substrate is three-dimensional, such as a cuboid, a cylinder, a cubic, etc. In some forms, the substrate is cuboid in shape and has a first dimension (i.e., width), a second dimension (i.e., length), and a third dimension (i.e., thickness). For example, the substrate has a width ranging from 100 μm to 1 mm, a length ranging from 100 μm to 1 mm, and a thickness ranging from about 10 nm to about 1 mm.

In some forms, the substrate of the working electrode is formed from a material capable of conducting an electric current, which can be organic or inorganic in nature, as long as it is able to conduct electrons. Examples of suitable materials forming the conductive substrate include, but are not limited to, a carbon-based material, a metal, a metal alloy, a conductive polymer, or a metal oxide, or a combination thereof. For example, the conductive substrate of the working electrode is formed from glassy carbon, gold.

In some forms, the conductive substrate of the working electrode is formed from a carbon-based material. Examples of suitable carbon-based materials forming the conductive substrate include, but are not limited to, glassy carbon, conducting polymers (in the form of films or fibers) carbon cloth, carbon paper, carbon screen printed electrodes, carbon paper, carbon black, carbon powder, carbon fiber, singe-walled carbon nanotubes, double-walled carbon nanotubes, multi-walled carbon nanotubes, carbon nanotube arrays, diamond-coated conductors, and mesoporous carbon. Additional exemplary carbon-based materials are graphene, graphite, uncompressed graphite worms, delaminated purified flake graphite, high performance graphite and carbon powders, highly ordered pyrolytic graphite, pyrolytic graphite, and polycrystalline graphite.

In some forms, the conductive substrate of the working electrode is formed from a metal. Examples of suitable metals forming the conductive substrate include, but are not limited to, gold, chromium, platinum, iron, nickel, copper, silver, stainless steel, mercury, tungsten, and other metals suitable for electrode construction. In some forms, the metals forming the conductive substrate are not in the form of nanomaterials, such as gold nanoparticles.

In some forms, the conductive substrate of the working electrode is formed from a metal alloy. Examples of suitable metal alloys forming the conductive substrate can be an alloy of any two or more of the metals described above, such as gold/chromium.

In some forms, the conductive substrate of the working electrode is formed from a metal oxide or metal sulfide. Examples of suitable metal oxides and metal sulfides forming the conductive substrate include, but are not limited to, nanoporous titanium oxide, tin oxide coated glass, glass, cerium oxide particles, molybdenum sulfide, boron nitride nanotubes, aerogels modified with a conductive material such as gold, solgels modified with conductive material such as carbon, ruthenium carbon aerogels, and mesoporous silicas modified with a conductive material such as gold.

In some forms, the conductive substrate of the working electrode is formed from a semiconductor optionally doped by with other elements. Examples of suitable semiconductors forming the conductive substrate include, but are not limited to, silicon and germanium, which can be doped (i.e., the intentional introduction of impurities into an intrinsic semiconductor for the purpose of modulating its electrical and structural properties) with other elements. The semiconductors can be doped with phosphorus, boron, gallium, arsenic, indium or antimony, or a combination thereof.

In some forms, the substrate of the working electrode is formed from a material that is not electrically conductive. Examples of suitable materials forming the non-conductive substrate include, but are not limited to, plastics, paper, textiles, or films. For example, the substrate of the working electrode is formed from a non-conductive film.

### 2. Counter and Reference Electrodes

The electrochemical sensor may include one or more additional electrodes, such as a counter electrode and/or a reference electrode. In some forms, the electrochemical sensor does not include a reference electrode, such as a Ag/AgCl reference electrode.

The counter electrode, which is often also called the auxiliary electrode, in the electrochemical sensor is an electrode used in a three electrode electrochemical cell for voltametric analysis or other reactions in which an electric current is expected to flow. Exemplary counter electrodes suitable for use in the electrochemical sensor include, but not limited to, gold, copper, carbon, and conducting polymer.

The reference electrode in the electrochemical sensor is an electrode having a maintained potential, used as a reference for measurement of other electrodes. Exemplary reference electrodes suitable for use in the electrochemical sensor include, but not limited to, silver, silver chloride, silver/silver chloride, gold, copper, carbon, and conducting polymer.

In some forms, the electrochemical sensor disclosed herein includes a working electrode as describe above, a platinum counter electrode, and a Ag/AgCl reference electrode.

Each of the counter and reference electrodes can have any suitable shape and dimensions but are generally similar to that of the substrate of the working electrode. For example, the electrochemical sensor includes a counter and a length ranging from about 2 mm to about 20 mm, a width ranging from about 0.1 mm to about 2 mm, and a thickness ranging from about 10 mm to about 2 mm.

### 3. Point-of-Care Device

The electrochemical sensor may be integrated on a support for point-of-care testing (also referred to herein as “point-of-care device”). For example, the working electrode and optionally the counter and/or reference electrode can be integrated on a microfluidic chip for point-of-care testing of target analytes in a biological sample, such as miRNA in blood or saliva.

The working electrode and optionally the counter and/or reference electrode can be integrated on the support using any suitable methods, such as by screen-printing or inkjet-printing using a suitable material as described above for the working, counter, and reference electrodes. Such microfluidic chips may be disposed after one use or multiple uses.

The support of the point-of-care device is typically electrically non-conductive, and has a planar surface. Examples of materials forming the support for the point-of-care device include, but are not limited to, plastics, paper, textiles, a wound dressing or bandage, a medical implant such as catheter, or glass. For example, the support of the point-of-care device is formed from paper or a plastic.

In some forms, the support of the point-of-care device may also be the substrate of the working electrode. For example, in the point-of-care device, the hybrid material disclosed herein is directly deposited on a support (such as by screen-printing or inkjet-printing), along with a counter and/or reference electrode deposited on the same support.

In some forms, the point-of-care device contains more than one working electrode/hybrid material, and optionally more than one counter electrode and/or more than one reference electrode, which is capable of performing multiplex detection. In these forms, the working electrodes/hybrid materials can contain the same or different biorecognition elements, wherein in the later, the device is capable of detecting more than one analyte at the same time. For example, at least one of the working electrodes/hybrid materials contains a first biorecognition element and one or more of the working electrodes/hybrid materials contains a second biorecognition element, where the first biorecognition element is different from the second biorecognition element.

In forms where a working, a counter, and a reference electrode are integrated on a microfluidic chip, the hybrid material, counter, and reference electrodes are arranged such that they are not in contact with one another. The microfluidic chip containing the working, counter, and reference electrodes may also contain one or more electrically conductive connects to allow electrons to flow between the working, counter, and/or reference electrodes and a reader capable of providing a voltage and measuring a current generated on the working electrode. The conductive connects can be formed using any suitable electrically conductive material, such as any of those described above for forming the working, counter, and reference electrodes, for example, gold, silver, or carbon-based material.

### 4. Other Components

Optionally, the electrochemical sensor further includes a reader. The reader can contain an acquisition component and optionally a display component.

For example, the electrochemical sensor is in the format of a point-of-care device, which includes a working electrode or hybrid material, a counter electrode, and a reference electrode screen-printed on a plastic or paper support; the support also contains electrically conductive connects printed thereon that electrically connect the working, counter, and reference electrodes to the acquisition component of the reader when attached thereto. Typically, the electrochemical sensor is portable, and the support containing the electrodes printed thereon may be attached or disconnected from the reader as needed.

The acquisition component of the reader may be a potentiostat, which is capable of providing a voltage and measuring a current generated on the working electrode. Optionally, the acquisition component is capable of connecting to a software installed on a display component that converts data into a graph, chart or table, for a target analyte such as a miRNA of interest.

Optionally, the reader includes a display component which may be a screen capable of displaying a current reading measured by the acquisition component of the reader. In some other forms, the reader does not include a display component, and instead, the reader can be connected to a smartphone, tablet, laptop, and/or monitor to display current data measured by the acquisition component of the reader.

### III. Methods of Making

The disclosed hybrid materials are prepared by covalently attaching biorecognition elements on one or more surfaces of MXene sheets via a suitable chemical linker. Preferably, the biorecognition elements are attached to the surface(s) of MXene sheets using copper-free click chemistry, which provides highly stable attachment and high loading density of the biorecognition elements, under ambient conditions (at room temperature and 1 atm, without the use of catalysts).

Generally, the preparation method includes: (a) treating MXene sheets having a plurality of surface hydroxyl groups with a clickable group functionalized silane or phosphonic acid having the structure of

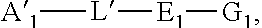

wherein A′₁ can be

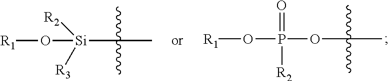

R₁ can be a C₁-C₄ unsubstituted alkyl; R₂ and R₃ can be independently

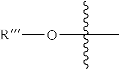

or a C₁-C₄ unsubstituted alkyl; R′″ can be hydrogen or a C₁-C₄ unsubstituted alkyl; L′ can be

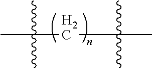

or

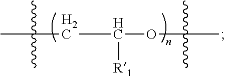

each occurrence of n can be independently an integer from 1 to 20, for example 1, 2, 2, 4, 5, 6, 7, 8, 9, 10, 11, 12, 13, 14, 15, 16, 17, 18, 19, 20; R′₁ can be hydrogen or C₁-C₄ unsubstituted alkyl (e.g., methyl, ethyl, etc.); E₁ can be a bond, a peptide, or

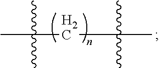

and G₁ can be a clickable group, to form clickable group functionalized MXene sheets, and (b) reacting the clickable group functionalized MXene sheets with a clickable group functionalized recognition element G₂-B₁, wherein B₁ is the recognition element and G₂ is a corresponding clickable group, to form the hybrid material.

In some forms, the clickable group functionalized silane or phosphonic acid in step (a) can have the structure of

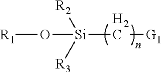

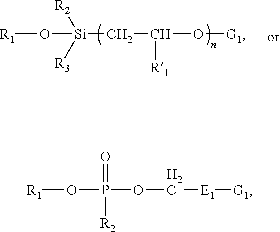

wherein R₁, R₂, R₃, R′₁, n, E₁, and G₁ can be as defined above.

The corresponding clickable group G₂ of the clickable group functionalized recognition element in step (b) can be any suitable functional group capable of reacting with the clickable group G₁ on the MXene via Click Chemistry.

For example, G₁ can be

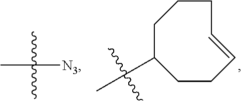

or

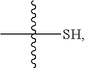 [SMILES: *S]

### and G₂ can be

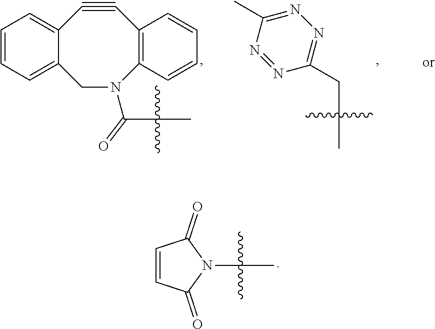

### Alternatively, G₁ can be

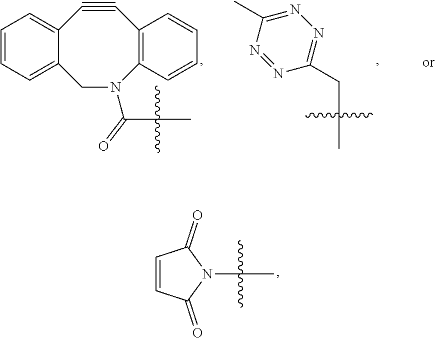

### and G₂ can be

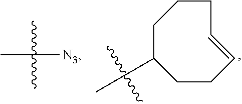

or

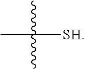 [SMILES: *S]

For example, G₁ can be

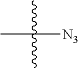 [SMILES: *N=[N+]=[N-]]

### and G₂ can be

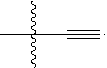 [SMILES: *C#C]

### Alternatively, G₁ can be

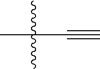 [SMILES: *C#C]

### and G₂ can be

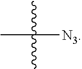 [SMILES: *N=[N+]=[N-]]

Typically, in step (a), the mole amount of clickable group functionalized silane is in excess to the mole amount of surface hydroxyl groups of the MXene sheets, to ensure a high conversion rate of the hydroxyl groups. For example, following step (a), at least 50%, at least 60%, at least 70%, at least 80%, or at least 90% of the hydroxyl groups on the surface of the MXene sheets are functionalized with the clickable group, such as

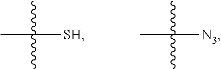

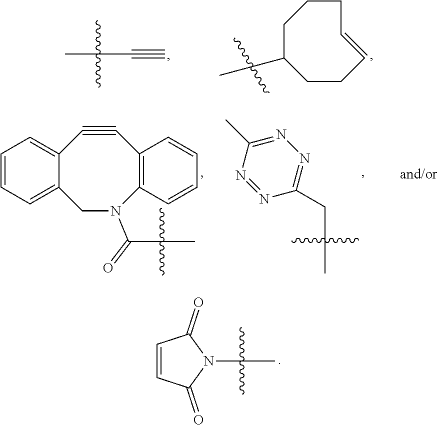

Similarly, in step (b), the clickable group functionalized biorecognition element is in excess to the clickable group functionalized MXene sheets, to ensure high loading of the biorecognition element following step (b). For example, the clickable group functionalized MXene sheets and clickable group functionalized biorecognition element has a weight ratio ranging from 10:1 to 1:10, such as 1:5 to 1:2. For example, following step (b), at least 50%, at least 60%, at least 70%, at least 80%, or at least 90% of the clickable group on the surface of the clickable group functionalized MXene sheets are covalently conjugated with the biorecognition elements. The hybrid material produced in step (b) has a loading of biorecognition elements of at least 1 mole based on the amount of the MXene sheets.

Step (a) may be performed in a solvent. In some forms, step (a) can be performed without a solvent. Examples of suitable solvents for use in step (a) include, but are not limited to, ethanol. The reaction between the MXene sheets and clickable group functionalized silane can be performed in the solvent, such as any one of those described above, or without a solvent, at room temperature (20° C.-25° C. at 1 atm) for a time period ranging from about 30 mins to about 60 hours, from about 30 mins to about 48 hours, from about 30 mins to about 24 hours, from about 30 mins to about 12 hours, from about 30 mins to about 8 hours, from about 24 hours to about 60 hours, from about 24 hours to about 48 hours, or from about 36 hours to about 48 hours.

Similarly, step (b) may be performed in a solvent, such as water, or without a solvent. In some forms, the reaction between the clickable group functionalized MXene sheets and clickable group functionalized biorecognition element can be performed in the solvent, such as water, at room temperature for a time period ranging from about 30 mins to about 18 hours, from about 30 mins to about 12 hours, from about 30 mins to about 8 hours, from about 30 mins to about 3 hours, from about 1 hour to about 18 hours, from about 1 hour to about 12 hours, from about 1 hours to about 8 hours, from about 1 hour to about 3 hours, from about 6 hours to about 18 hours, from about 6 hours to about 15 hours, or from about 9 hours to about 12 hours.

Optionally, step (a) and/or (b) is performed under agitation, such as by stirring. Technique for applying agitation for chemical reactions are known, such as mechanical stirring using a stirring rod, magnetic stirring, shaking/inverting on a rocker, etc.

The method may further include a purification step after step (a) and prior to step (b), and/or after step (b), to remove unreacted components, such as unreacted clickable group functionalized silane or phosphonic acid. The purification can be performed using any known technique. For example, the method further includes washing and centrifugating the clickable group functionalized MXene sheets, after step (a) and prior to step (b), to remove unreacted clickable group functionalized silane or phosphonic acid; and/or washing the hybrid material after step (b) to remove unreacted clickable group functionalized biorecognition element.

The hybrid material can be deposited on a surface of a substrate, such as a glassy carbon or non-conductive film, using methods known in the art, such as drop-casting, spin-coating, sputter-coating, etc., to obtain the working electrode of the disclosed electrochemical sensors.

The electronic components, such as working/hybrid material, counter, and reference electrodes and conductive connects, can be deposited on a non-conductive support using methods known in the art, such as screen-printing and inkjet technology.

### IV. Methods of Using

Electrochemical sensors incorporating the hybrid materials disclosed herein can be used for non-invasive detection of the absence, the presence, and/or the concentration of biomarkers, such as nucleic acids, proteins, pathogens, components of pathogens, hormones, carbohydrates, cells, extracellular vesicles, and viruses, in a biological sample, such as blood serum, plasma, urine, saliva, etc. and extracts therefrom. Exemplary analytes that can be detected in a sample include, but are not limited to peptides, proteins, nucleic acids (such as DNA and RNA), pathogens, components of pathogens, hormones, carbohydrates, cells, extracellular vesicles, and viruses.

In some embodiments, the sample can include RNA, such as total RNA or size selected RNA (e.g., small RNA). For example, the sample can be enriched for miRNAs. In some embodiments, the sample includes RNA selected from small nucleolar RNA, messenger RNA (mRNA), tRNA, small interfering RNA (siRNA), microRNA, and antisense RNA.

In preferred embodiments, the sample includes one or more miRNAs. In some embodiments, the miRNAs can be associated with a disease or disorder such as cancer, cardiovascular diseases, liver diseases, sepsis, infectious diseases (or causative agents thereof), genetic disorders, metabolic disorders, and neurodegenerative diseases.

Exemplary miRNAs that can be detected by any of the disclosed methods include, without limitation, miR-141, miR-21, miR-122, miR-192, miR-21, miR-223, miR-375, miR-30a, miR-33a, miR-34a, miR-16, miR-155, miR-132, miR-27a, miR-150, miR-199, miR-200, miR-17, miR-214, miR-9, miR-29a, miR-212, miR-214, miR-497, miR-378, miR-320, miR-222, miR-106a, miR-92, miR-20, miR-23, miR-18, miR-126, and Cel-miR-39.

The detection result obtained using the disclosed methods can help with diagnosing a presence or absence of a disease, such as diabetes, a malignant disease, neurological disease, alcoholism, infection (viral, bacterial or fungal), immune response (allergy, asthma, immunosuppression), and cardiovascular disease. In some forms, the detection result obtained using the disclosed methods can help with prognosis of a disease or a disease course, such as diabetes, malignant disease, neurological disease, alcoholism, viral infections, bacterial infections, and cardiovascular disease.

The sample can be isolated or derived from any suitable sample from the subject. Thus, in some forms, the methods can involve obtaining a nucleic acid sample from a subject. Exemplary samples include blood (such as peripheral blood), plasma, serum, urine, saliva, mucosal excretions, sputum, stool, spinal fluid, amniotic fluid, lymph fluid, vitreous, urine, tears, perspiration, semen and the like. Isolation and extraction of the nucleic acids may be performed through collection of cells, tissues or bodily fluids using a variety of techniques. In some forms, collection may involve aspiration of a bodily fluid from a subject using a syringe. In other embodiments, collection may involve pipetting or direct collection of fluid into a collecting vessel.

In some embodiments, the sample contains nucleic acids (e.g., RNA) isolated or derived from exosomes or other extracellular vesicles. Extracellular vesicles (EVs) are a heterogeneous collection of membrane-bound structures with complex cargoes including proteins, lipids, and nucleic acids. EV subtypes include ectosomes, microvesicles (MV), microparticles, exosomes, oncosomes, apoptotic bodies (AB), and tunneling nanotubes (TNT) (Yáñez-Mó, et al., J Extracell Vesicles. 4: 27066 (2015)).

The nucleic acid sample may be isolated and extracted using a variety of techniques known in the art. In some cases, cell free DNA/RNA may be isolated, extracted and prepared using commercially available kits such as Trizol®.

Generally, nucleic acids are extracted and isolated from samples through a partitioning step in which cell free DNAs, as found in solution, are separated from cells and other non-soluble components of the sample. Partitioning may include, but is not limited to, techniques such as centrifugation or filtration. In other cases, cells are not partitioned from cell free DNA first, but rather lysed. In this example, the genomic DNA of intact cells is partitioned through selective precipitation. DNA or other nucleic acids may remain soluble and may be separated from insoluble genomic DNA and extracted. Generally, after addition of buffers and other wash steps specific to different kits, DNA may be precipitated using isopropanol precipitation. Further clean up steps may be used such as silica-based columns to remove contaminants or salts. General steps may be optimized for specific applications.

Isolation and purification of cell free DNA may be accomplished using any means, including, but not limited to, the use of commercial kits and protocols provided by companies such as Sigma Aldrich, Life Technologies, Promega, Affymetrix, IBI or the like. Kits and protocols may also be non-commercially available.

In some forms, particularly where cellular RNA is the desired nucleic acid for analysis, the RNA can be isolated from the cell lysate. In some forms, the genomic DNA is removed from the cell lysate, and the cell lysate, including total cellular RNA is utilized as the starting material for reverse transcription. In some forms, isolation of total RNA and removal of genomic DNA are combined.

Methods and kits for facilitating RNA isolation, and/or removal of genomic DNA are known in the art and can be used or modified as known in the field of nucleic acid amplification to facilitate preparation of RNA for reverse transcription. An exemplary kit is Rneasy® Plus Micro Kit (Qiagen). The process typically includes spinning cell or tissue lysates through spin columns to remove genomic DNA. Next, total RNA is purified using a second spin column. In some embodiments, an RNA carrier, such synthetic poly(A) RNA, can added to the lysis buffer before homogenizing the cells. Mild lysis buffer can include one or more detergents such as TRITON®-X100, IGEPAL CA-630, NP40, TWEEN® 20 at a concentration of about 0.01 to about 2%. See, e.g., U.S. Pat. No. 10,017,761.

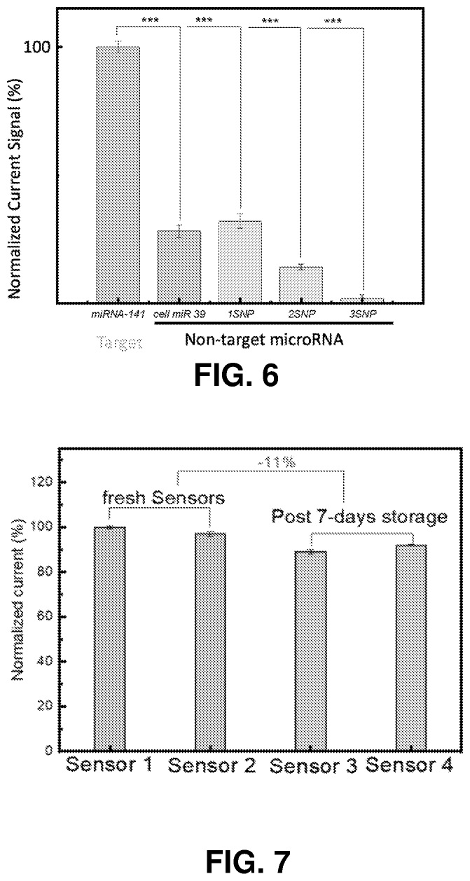

Compared to other methods for biomarker detection, methods using electrochemical sensors incorporating the disclosed hybrid material offer high sensitivity and specificity while being more cost-effective, time-effective, facile, isothermal and enzyme-free. For example, using the electrochemical sensor disclosed herein, detection of biomarkers in liquid biological samples can be achieved with high sensitivity (down to attomolar limit of detection, such as down to about 40 aM, or down to about 180 fM, of the target analyte), high reproducibility, a wide linear dynamic range (from attomolar to nanomolar, such as from about 40 aM to about 1 nM, or from about 0.1 fM to about 1 nM of target analyte), and high target specificity towards the target analyte (see, e.g., FIG. 6). It is believed that the disclosed hybrid material is the first demonstration of a MXene-biorecognition element hybrid material, which, when used in an electrochemical sensor, allows amplification-free, enzyme-free, and target-specific detection of a variety of analytes, such as nuclei acids (e.g., miRNA), proteins, pathogens, and components of pathogens.

Typically, the detection methods disclosed herein generate an electrochemical “turn-on” signal, which is desirable for detection of low-quantity target analytes, such as miRNA. For example, sensing using the disclosed sensors relies on the specific interaction between the biorecognition element and target analyte, such as sequence-specific hybridization between the immobilized PNA probes and target microRNA through Watson-Crick base-pairing, which in turn facilitates intercalation of a redox probe (such as methylene blue) in the biorecognition element-target analyte pair to generate an electrochemical signal. This results in a target-specific, sensitive, and quantitative electrochemical ‘turn-on’ signal correlating to the amount of target analytes captured, without the need for expensive enzymes, amplification strategies or other nanomaterial-based labels.

Generally, the method for detecting the absence, the presence, and/or the concentration of an analyte of interest in a liquid biological sample includes: (i) contacting the working electrode of the electrochemical sensor disclosed herein with the biological sample; (ii) optionally contacting the working electrode of the sensor with a redox reporter solution; and (iii) measuring an electrochemical signal generated by the sensor. Steps (i) and (ii) can be performed simultaneously, in some forms.

The biological sample may be a bodily fluid (such as whole blood, plasma, serum, saliva, interstitial fluid, nasal fluid, tears, breast milk, mucus, sputum, bronchial alveolar lavage (BAL), bronchial wash (BW), cerebrospinal fluid (CSF), or urine), an extract of a bodily fluid, or a non-bodily fluid (e.g., a buffer or an environmental fluid, such as water from rivers, lakes, reservoirs, wetlands, etc.), or a combination thereof. Exemplary buffers include, but are not limited to, phosphate buffer solution (PBS), salt water, MES buffer, Bis-Tris buffer, ADA, ACES, PIPES, MOPSO, Bis-Tris propane, BES, MOPS, TES, HEPES, DIPSO, MOBS, TAPSO, Trizma, HEPPSO, POPSO, TEA, EPPS, Tricine, Gly-gly, Bicine, HEPBS, TAPS, AMPD, TABS, AMPSO, CHES, CAPSO, AMP, CAPS, or CABS, or a combination thereof. The buffer can have a pH between 3 and 8.5, preferably a pH of 7.4.

The volume of biological sample for detection can be from about 0.1 μL to about 1 mL. In some forms, the volume of biological sample is from about 0.1 μL to about 100 μL, from about 0.1 μL to about 50 μL, from about 0.1 μL to about 30 μL, from about 1 μL to about 30 μL, from about 1 μL to about 20 μL, from about 1 μL to about 10 μL, or from about 1 μL to about 5 μL, such as ≤20 μL, ≤10 μL, or ≤5 μL.

Typically, in step (i), the working electrode is in contact with the biological sample for a time period ranging from about 1 min to about 4 hours, from about 1 min to about 2 hours, from about 1 min to about 1 hour, from about 1 min to about 30 mins, from about 1 min to about 20 mins, from about 1 min to about 10 mins, from about 10 mins to about 40 mins, from about 15 mins to about 35 mins, or from about 20 mins to about 30 min, optionally at room temperature. Optionally, the working electrode is washed with a suitable solvent, such as water or buffer, for one or more times after step (i) and prior to step (ii).

In step (ii), the working electrode is in contact with the redox reporter solution for a time period ranging from about 1 min to about 10 mins or from about 1 min to about 5 mins, at room temperature. The redox reporter can be any molecule capable of accepting or donating an electron when a suitable voltage is applied thereto. Examples of redox reporter for use in the disclosed method include, but are not limited to, methylene blue, ferrocene, ferrocyanide, ferricyanide, tris(bipyridine)ruthenium(II) chloride, hexaammineruthenium(III) chloride, or anthraquinone, or a combination thereof. The redox reporter in the redox reporter solution can have any suitable concentration, such as in a range from 0.1 μM to 1 M. Optionally, the working electrode is washed with a suitable solvent, such as water or buffer, for one or more times after step (ii) and prior to step (iii).

In step (iii), the electrochemical signal generated by the sensor can be measured using any suitable method, such as cyclic voltammetry, differential pulse voltammetry, square wave voltammetry, amperometry, or electrochemical impedance spectroscopy, or a combination thereof, for example, using cyclic voltammetry and/or differential pulse voltammetry. When cyclic voltammetry or differential pulse voltammetry is used for measuring the electrochemical signal generated by the sensor, a voltage is applied to the working electrode from a first value to a second value, and a current is generated across the range of applied voltage. Typically, a peak current (i.e., maximum current generated across the applied voltage) is used as the electrochemical signal. The voltage at which a peak current is expected can be estimated based on the specific redox reporter, the specific electrochemical technique, and the specific reference electrode used in the method. For example, when methylene blue and cyclic voltammetry are used in the detection method, a peak current is expected at about −0.18 to −0.2 V vs. Ag/AgCl. When methylene blue and differential pulse voltammetry are used in the detection method, a peak current is expected at about −0.3 to −0.35 V vs. Ag/AgCl.

Optionally, the detection method further includes: (iv) contacting the working electrode of the sensor with the redox reporter solution; and (v) measuring a background signal generated by the sensor, wherein steps (iv) and (v) are performed prior to step (i). In step (iv), the working electrode is in contact with the redox reporter solution for a time period ranging from about 1 min to about 10 mins or from about 1 min to about 5 mins, at room temperature. Optionally, the detection method further includes washing the working electrode one or more times after step (iv) and prior to step (v). Typically, step (v) is performed in a buffer solution, optionally wherein the buffer solution is a TE buffer at physiological pH (i.e., pH 7.4), for measuring the background signal. The background signal is typically measured using the same method as the electrochemical signal measured in step (iii).

Typically, the background signal is the current value at the voltage where a peak current is expected for the redox reporter. The background signal can be used to determine the absence, presence, or specific concentration of the target analyte in the biological sample. For example, the difference between the absolute value of the electrochemical signal (peak current) and the absolute value of the background signal (current) is indicative of the absence, presence, or concentration of the target analyte. For example, an electrochemical signal that is more intense compared to the background signal is indicative of the presence of the analyte of interest in the biological sample; an electrochemical signal that is similarly (within 30%) or less intense compared to the background signal is indicative of the absence of the analyte of interest in the biological sample; and different concentrations of the analyte of interest in the biological sample can be determined based on the intensity level of the electrochemical signal compared to the background signal. For determining the concentration of the target analyte, a calibration curve may be first established, which is exemplified in the Examples below. Steps (iv) and (v) may be performed by a user performing the detection method or performed by a manufacturer that then provides a background signal for the user to compare with the electrochemical signal measure in step (iii). In some forms, a standard calibration curve which can be used for determining the concentration of the target analyte is also provided by a manufacturer.

The examples below demonstrate use of the disclosed electrochemical sensors to achieve robust, simple, and cost-effective detection of target analytes, which is amenable for point-of-care.

The disclosed compositions and methods can be further understood through the following numbered paragraphs.

1. A hybrid material comprising a plurality of MXene sheets having biorecognition elements conjugated thereto, wherein the biorecognition elements comprise a binding partner to an analyte of interest.

2. The hybrid material of paragraph 1, wherein the biorecognition elements are conjugated to one or more surfaces of the MXene sheets via a linker, and wherein the linker is represented by the formula:

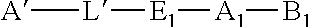

wherein A′ is

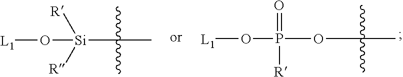

L₁ is an attachment point to the surface of MXene;
R′ and R″ are independently

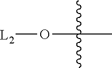

or a C₁-C₄ unsubstituted alkyl;
L₂ is an attachment point to the surface of MXene, a C₁-C₄ unsubstituted alkyl, or hydrogen;
L′ is

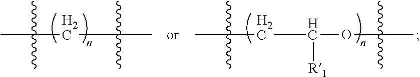

each occurrence of n is independently an integer from 1 to 20;
R′₁ is hydrogen or C₁-C₄ unsubstituted alkyl (e.g., methyl, ethyl, etc.);
E₁ is a bond, a peptide, or

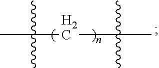 [SMILES: *C*]

A₁ is a moiety formed by click chemistry; and
B₁ is the biorecognition element,

Optionally wherein the linker is represented by the formula:

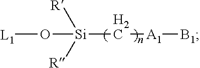

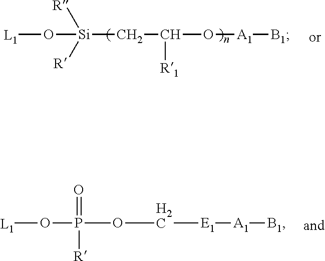

optionally wherein A₁ is

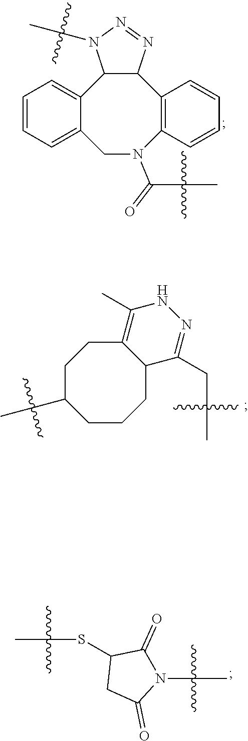 [SMILES: *C(=O)N1Cc2ccccc2C2C(N=NN2*)c2ccccc21 ; *CC1=NNC(C)=C2CCC(*)CCCC12 ; *SC1CC(=O)N(*)C1=O]

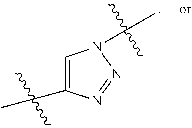 [SMILES: *c1cn(*)nn1]

3. The hybrid material of paragraph 1 or 2, wherein one of R′ and R″ is

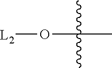

and L₂ is an attachment point to the one or more surfaces of the MXene sheets, or wherein both R′ and R″ are

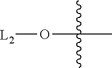

and L₂ is an attachment point to the one or more surfaces of the MXene sheets..

4. The hybrid material of any one of paragraphs 1-3, wherein the biorecognition element is a nucleic acid, a peptide, or a protein (e.g., an antibody, a nanobody, an antibody fragment, or an enzyme), optionally wherein the biorecognition element is a peptide nucleic acid.

5. The hybrid material of any one of paragraphs 1-4, wherein the analyte of interest is a biomarker,
optionally wherein the analyte of interest is a nuclei acid, a protein, a pathogen, a component of a pathogen, a hormone, a carbohydrate, a cell, an extracellular vesicle, or a virus,
optionally wherein the analyte of interest is a miRNA.

6. The hybrid material of any one of claims 1-5, wherein each MXene sheet comprises
a layered body represented by Mₙ₊₁Xₙ, wherein n is an integer from 1 to 4, each M is a metal of Group 3, 4, 5, 6, or 7, and each X is a carbon atom or a nitrogen atom; and
optionally one or more surface functional groups.

7. The hybrid material of paragraph 6, wherein each M is selected from the group consisting of Ti, Zr, Hf, V, Nb, Ta, Cr, and Mo.

8. The hybrid material of paragraph 6 or 7, wherein the functional group(s) is/are hydroxyl, oxide, halogen, or carbonyl, or a combination thereof.

9. The hybrid material of any one of paragraphs 1-8, wherein the MXene sheets are Ti₃C₂ nanosheets and optionally with one or more surface hydroxyl groups.

10. A sensor comprising a working electrode,
wherein the working electrode comprises a substrate and the hybrid material of any one of paragraphs 1-9 deposited thereon.

11. The sensor of paragraph 10, wherein the hybrid material forms a coating on one or more surfaces of the substrate, optionally wherein the coating is a porous coating.

12. The sensor of paragraph 10 or 11, wherein the substrate is formed from a conductive material, optionally wherein the conductive material is carbon-based material, a metal, or a metal alloy, optionally wherein the substrate is formed from glassy carbon or gold,

13. The sensor of any one of paragraphs 10-12, wherein the substrate is formed from a non-conductive film.

14. The sensor of any one of paragraphs 10-13, further comprising a counter electrode and optionally a reference electrode, optionally wherein the counter electrode is platinum, optionally wherein the reference electrode is Ag/AgCl.

15. A method of making the hybrid material of any one of paragraphs 1-9, comprising:
(a) treating MXene sheets having a plurality of surface hydroxyl groups with a clickable group functionalized silane or phosphonic acid having the structure of

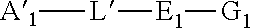

wherein A′₁ is

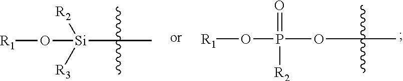

R₁ is a C₁-C₄ unsubstituted alkyl;
R₂ and R₃ are independently

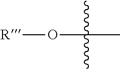

or a C₁-C₄ unsubstituted alkyl;
R′″ is hydrogen or a C₁-C₄ unsubstituted alkyl;
L′ is

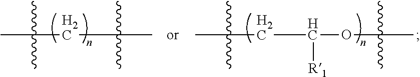

each occurrence of n is independently an integer from 1 to 20, such as from 1 to 6;
R′₁ is hydrogen or C₁-C₄ unsubstituted alkyl (e.g., methyl, ethyl, etc.);
E₁ is a bond, a peptide, or

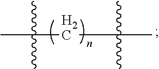

and
G₁ is a clickable group,
to form clickable group functionalized MXene sheets, and
(b) reacting the clickable group functionalized MXene sheets with a clickable group functionalized biorecognition element G₂-B₁, wherein B₁ is the biorecognition element and G₂ is a corresponding clickable group capable of reacting with the clickable group of the clickable group functionalized MXene sheets via Click Chemistry,
to form the hybrid material,
optionally wherein the clickable group functionalized silane or phosphonic acid has the structure of

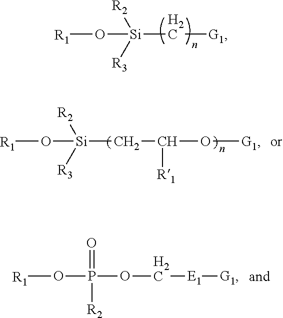

optionally wherein G₁ and G₂ are selected from the group consisting of

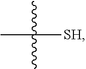 [SMILES: *S]

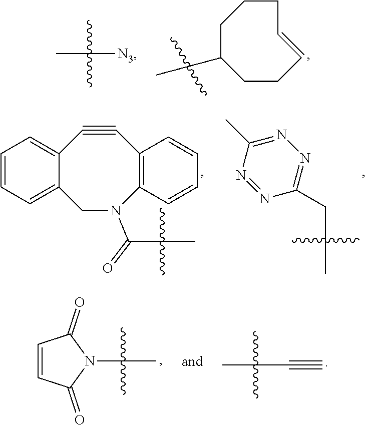

16. The method of paragraph 15, wherein in step (a), the mole amount of clickable group functionalized silane or phosphonic acid is in excess to the mole amount of surface hydroxyl groups.

17. The method of paragraph 15 or 16, step (a) is performed in a solvent, such as ethanol, or without a solvent.

18. The method of any one of paragraphs 15-17, wherein step (a) is performed at room temperature (20° C.-25° C. at 1 atm) for a time period ranging from about 30 mins to about 60 hours, from about 30 mins to about 48 hours, from about 30 mins to about 24 hours, from about 30 mins to about 12 hours, from about 30 mins to about 8 hours, from about 24 hours to about 60 hours, from about 24 hours to about 48 hours, or from about 36 hours to about 48 hours.

19. The method of any one of paragraphs 15-18, further comprising washing and centrifugating the clickable group functionalized MXene sheets, after step (a) and prior to step (b), to remove unreacted clickable group functionalized silane or phosphonic acid.

20. The method of any one of paragraphs 15-19, wherein in step (b), the weight ratio of clickable group functionalized MXene sheets to clickable group functionalized biorecognition element is in a range from 10:1 to 1:10 or from 1:5 to 1:2.

21. The method of any one of paragraphs 15-20, wherein step (b) is performed in a solvent, such as water.

22. The method of any one of paragraphs 15-21, wherein step (b) is performed at room temperature for a time period ranging from about 30 mins to about 18 hours, from about 30 mins to about 12 hours, from about 30 mins to about 8 hours, from about 30 mins to about 3 hours, from about 1 hour to about 18 hours, from about 1 hour to about 12 hours, from about 1 hours to about 8 hours, from about 1 hour to about 3 hours, from about 6 hours to about 18 hours, from about 6 hours to about 15 hours, or from about 9 hours to about 12 hours.

23. The method of any one of paragraphs 15-22, further comprising washing the hybrid material after step (b) to remove unreacted clickable group functionalized biorecognition element.

24. The method of any one of paragraphs 15-23, wherein step (a) and/or (b) is performed under agitation, such as stirring.

25. A method of detecting the absence, the presence, and/or the concentration of an analyte of interest in a liquid biological sample, comprising
(i) contacting the working electrode of the sensor of any one of paragraphs 10-14 with the biological sample;
(ii) optionally contacting the working electrode of the sensor with a redox reporter solution; and
(iii) measuring an electrochemical signal generated by the sensor.

26. The method of paragraph 25, further comprising (iv) contacting the working electrode of the sensor with the redox reporter solution; and (v) measuring a background signal generated by the sensor, wherein steps (iv) and (v) are performed prior to step (i).

27. The method of paragraph 25 or 26, wherein the redox reporter is methylene blue, ferrocene, ferrocyanide, ferricyanide, tris(bipyridine)ruthenium(II) chloride, hexaammineruthenium(III) chloride, or anthraquinone, or a combination thereof.

28. The method of any one of paragraphs 25-27, wherein in step (iii), the electrochemical signal is measured using a method selected from the group consisting of cyclic voltammetry, differential pulse voltammetry, square wave voltammetry, amperometry, or electrochemical impedance spectroscopy, or a combination thereof.

29. The method of any one of paragraphs 26-28, wherein in step (v), the background signal is measured using the same method as the electrochemical signal measured in step (iii).

30. The method of any one of paragraphs 25-29, wherein each of step (iii) and step (v) is preformed in a buffer solution, optionally wherein the buffer solution is a TE buffer at physiological pH

31. The method of any one of paragraphs 25-30, wherein in steps (i), the working electrode is in contact with the biological sample for a time period ranging from about 1 min to about 4 hours, from about 1 min to about 2 hours, from about 1 min to about 1 hour, from about 1 min to about 30 mins, from about 1 min to about 20 mins, from about 1 min to about 10 mins, from about 10 mins to about 40 mins, from about 15 mins to about 35 mins, or from about 20 mins to about 30 min, optionally at room temperature.

32. The method of any one of paragraphs 25-31, wherein in each of step (ii) and step (iv), the working electrode is in contact with the redox reporter solution for a time period ranging from about 1 min to about 10 mins or from about 1 min to about 5 mins, at room temperature.

33. The method of any one of paragraphs 25-32, further comprising washing the working electrode one or more times after step (i) and prior to step (ii), after step (ii) and prior to step (iii), and/or after step (iv) and prior to step (v).

34. The method of any one of paragraphs 25-33, wherein the biological sample is (a) a bodily fluid, (b) an extract of a bodily fluid, or (c) a non-bodily fluid (e.g., a buffer or an environmental fluid), or a combination thereof, optionally wherein the bodily fluid is selected from the group consisting of whole blood, plasma, serum, saliva, interstitial fluid, nasal fluid, tears, breast milk, mucus, sputum, bronchial alveolar lavage (BAL), bronchial wash (BW), cerebrospinal fluid (CSF), and urine.

35. The method of any one of paragraphs 25-34, wherein the volume of the biological sample is ≤20 μL, ≤10 μL, or ≤5 μL.

36. The method of any one of paragraphs 25-35, wherein the sensor has a limit of detection down to about 40 aM, such as about 180 fM, of the analyte of interest.

37. The method of any one of paragraphs 25-36, wherein the sensor has a detection dynamic range from about 40 aM to about 1 nM, such as from about 0.1 fM to about 1 nM.

38. The method of any one of paragraphs 25-37, wherein the sensor has a shelf stability of at least 1 week, at least 1 month, at least 3 months, or at least 6 months, at 4° C.

### EXAMPLES

### Example 1. Peptide Nucleic Acid/MXene Hybrid Material for Ultrasensitive Enzyme-Free Electrochemical Detection of microRNA

### Materials and Methods

### Materials

All microRNA oligonucleotides were purchased from Integrated DNA technologies. Other chemical reagents including (3-Azidopropyl) triethoxysilane (Gelest 98%), Hexaammineruthenium (III) chloride [Ru(NH₃)₆]Cl₃, Phosphate buffer saline (PBS), Alumina powder (1, 0.3, and 0.05 micron), potassium chloride (KCl, 99.0%), and Nafion were purchased from Sigma Aldrich. BP Fluor 647 (Alexa Fluor®647 analog) was purchased from Biopharma. Ethanol and buffer salts were of the analytical grade. Millipore purified Deionized (DI) water was used throughout experiments. DBCO functionalized PNA probes were synthesized in our lab following solid phase peptide synthesis protocols using Fmoc chemistry. The TE buffer was purchased from Sigma Aldrich.

### Synthesis of DBCO Functionalized Peptide Nucleic Acid Probe

Bespoke PNA probes (either 7-mer or 17-mer, Table 1) were synthesized based on standard solid phase peptide chemistry protocols using Fmoc Chemistry on solid resin using an automated peptide synthesizer (Purepep Chorus, Gyros Protein Technologies). The PNA probes were modified at the C-terminal with two arginine residues to improve solubility in aqueous conditions. The N-terminal was modified with dibenzocyclooctyne (DBCO) to allow copper-free click chemistry with the azido-functionalized MXene surface. After synthesis, the probes were cleaved using a cleavage cocktail of TFA:TIS:H₂O, then precipitated in cold diethyl ether before purification by reverse-phase HPLC (Shimadzu). The pure products were freeze-dried and validated by mass spectroscopy.

Synthesis of Ti₃C₂Tx MXene

Ti₃C₂Tx MXene nanosheets were prepared following published protocols. Briefly, 3:3:3 ml of HCl, HF (47-51%), and cold DI water were mixed and stirred for 5 min at 200 rpm. 1 g of MAX powder was gradually added in the etchant solution while stirring using a Teflon magnetic bar. Cold DI water (3 ml) was added to the suspension. This suspension was etched for −17 h in an oil bath at 40 degrees at 500 rpm, followed by several cycles of centrifugation with DI water at 2600 rpm for 5 min until a pH of 5-6 was obtained. Next, LiCl (lg/35 ml DI) solution was added to the precipitant and magnetically stirred for 30 min. The solution was washed with DI water multiple times until the solution reached a pH of 5-6. Finally, the solution was centrifuged several rounds at 500 rpm for 10 min and the supernatant was collected as ultrathin Ti₃C₂-MXene nanosheets.

### Preparation of Azide (N-3)-Functionalized MXene

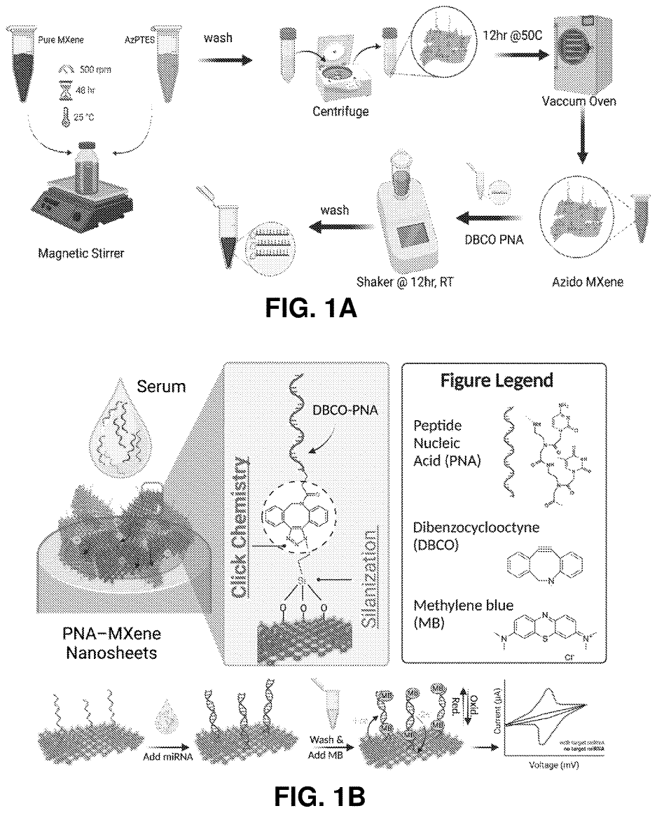

The as-synthesized ultrathin Ti₃C₂-MXene nanosheets were treated with (3-azidopropyl) triethoxysilane (AzPTES) to introduce azide functional groups by a silanization reaction as shown in FIG. 1A. In this experiment, 16 mg of Ti₃C₂ sheets was mixed with ethanol (8 ml). Next, the solution of the silane coupling agent (160 μl) was added dropwise to the suspension with magnetic stirring at room temperature at 500 rpm for 48 h. The silane was used in excess to ensure efficient reaction between the hydroxyl terminations of Ti₃C₂Tx and the AzPTES linker. After completion of the reaction, the suspension was washed three times with ethanol and water (1:1) by centrifugation at 3500 rpm to remove the unreacted silane coupling agents from AzPTES-Ti₃C₂Tx nanosheets. The final suspension of the AzPTES-Ti₃C₂Tₓ nanosheets was subsequently dried in a vacuum oven at 50° C. for 12 h. The obtained product was crushed and kept in a desiccator at room temperature for further use. The functionalized MXene was suspended in 1 ml of 0.1% Nafion solution and sonicated for 1 h to get a homogeneous solution.

### Click Reaction Between AzPTES-MXene and DBCO-PNA

Click chemistry was used as a high yield bio-orthogonal strategy to attach the PNA probe onto the MXene nanosheets. 5 mg of azido-functionalized MXene reacted with 19 mg of DBCO-functionalized PNA in DI water under stirring for 12 hr at room temperature. The product was washed three times with DI water to remove any unbound PNA molecules. The final product (PNA-MXene nanohybrid) was stored at RT in the dessicator for further use.

Preparation of PNA/Ti₃C₂Tx MXene-GCE (WE)

A glassy carbon electrode (GCE) was used as the working electrode in a three-electrode electrochemical setup to study the electrochemical performance and properties of AzPTES-functionalized MXene (f-MXene). For miRNA sensing, the GCE was polished with alumina slurry of 0.3 m and 0.05 μm in turn, then the GCE was sonicated in a mixture of acetone and DI water, followed by rinsing with DI water and drying under nitrogen flow. 4 μl of the MXene-PNA nanohybrid was drop-casted on the 2 mm GCE. The electrode was left to dry at room temperature in a laminar flow cabinet. The Ti₃C₂Tx-PNA showed high binding affinity to the electrode surface and high stability thereon. This bioelectrode (PNA-Ti₃C₂-GCE) was washed with TE buffer to remove the unbound PNAs. This electrode is hereafter referred to as the working electrode, The preparation of which is shown in FIG. 1B.

### Characterization

Ti₃C₂-MXene and f-Ti₃C₂-MXene nanosheets were characterized in their as-prepared state by Raman spectroscopy (LabRam Aramis, blue laser with excitation wavelength of 473 nm), X-ray photoelectron spectroscopy (XPS) (Kratos Analytical, AMICUS/ESCA 3400), Scanning electron microscope (SEM) (SEM Quattro), and Zetasizer Nano. Cyclic voltammetry was performed in the potential window 0.2 to −0.6 V at a scan rate of 100 mVs¹. Electrochemical impedance spectroscopy was exploited to monitor the changes in capacitance upon any binding event. Charge transfer resistance (R t) was monitored from 0.1 kHz to 100 kHz. A three-electrode electrochemical cell was used, which includes a modified GCE as the working electrode (2 mm² working area), platinum wire as the auxiliary electrode, and Ag/AgCl as the reference electrode in TE buffer (50 mM, pH 7.4).

### Results and Discussion

### PNA/MXene Conjugate Design

The PNA probe was designed as a 7-mer or 17-mer probe with sequences complimentary to hsa-miR-141 (Table 1) and a copper-free clickable end-moiety. The azide functionalized MXene was preprepared using surface modification with the functional silanization agent of AzPTES (FIG. 1A). As a result, the PNA/MXene was synthesized using the method of copper-free Click chemistry.

TABLE 1
microRNA/PNA sequences

| microRNA Name | Sequence |
|---|---|
| 7-mer PNA: | CATTTCT-Arg-Arg-DBCO |
| | (SEQ ID NO: 1) |
| 17-mer PNA | 3′ TGTGACAGACCATTTCT Arg- |
| | Arg-DBCO (SEQ ID NO: 2) |
| miR-141 | 5′ UAACACUGUCUGGUAAAGAUGG 3′ |
| | (SEQ ID NO: 3) |
| miR-141_1 SNP | 5′ UAACAAUGUCUGGUAAAGAUGG 3′ |
| | (SEQ ID NO: 4) |
| miR-141-2 SNPs | 5′ UAACAAUGUCUGGUAUAGAUGG 3′ |
| | (SEQ ID NO: 5) |
| miR-141-3 SNPs | 5′ UAAGACUGUCUGCUAAAGUUGG 3′ |
| | (SEQ ID NO: 6) |
| Cel-miR-39 | 5′ UCACCGGGUGUAAAUCAGCUUG 3′ |
| | (SEQ ID NO: 7) |

CATTTCT-ARG-ARG-DBCO is shown below.

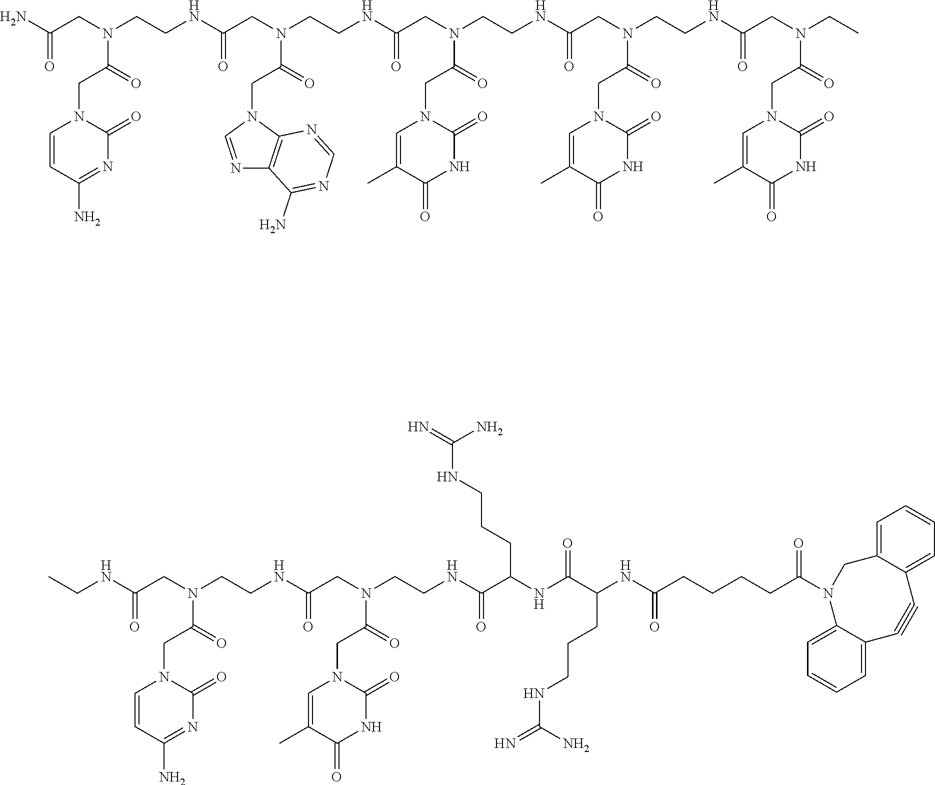

### Bioelectrode Design—PNA/MXene Hybrid on GCE Surface

As shown in FIG. 1A, the ethoxy groups in the AzPTES reacted with the OH⁻ groups on the MXene surface and thus MXene are provided with Azide groups (—N3). MXene is a stable and effective layer for immobilizing bioreagents to attain bioactivity. The working principle and electron transfer process of the Ti₃C₂Tx MXene modified electrode are shown in FIG. 1B. AzPTES-Ti₃C₂Tx MXene is mixed with 0.1% Nafion to facilitate fixing of the MXene to the GCE surface. The azide group on the AzPTES-Ti₃C₂Tx MXene provide PNA binding sites so that the PNA can be immobilized on the electrode surface via Cu-free click chemistry. Specific surface area and conductivity of the electrode is significantly improved. When the hybrids of the PNA probe-miRNA-141 formed, MB molecules were bound in between the hybridization chain. The concentration of miRNA-141 was directly correlated with the MB's associated oxidation peak current. This relationship can be used for accurate detection of miRNA.

### Materials Characterization

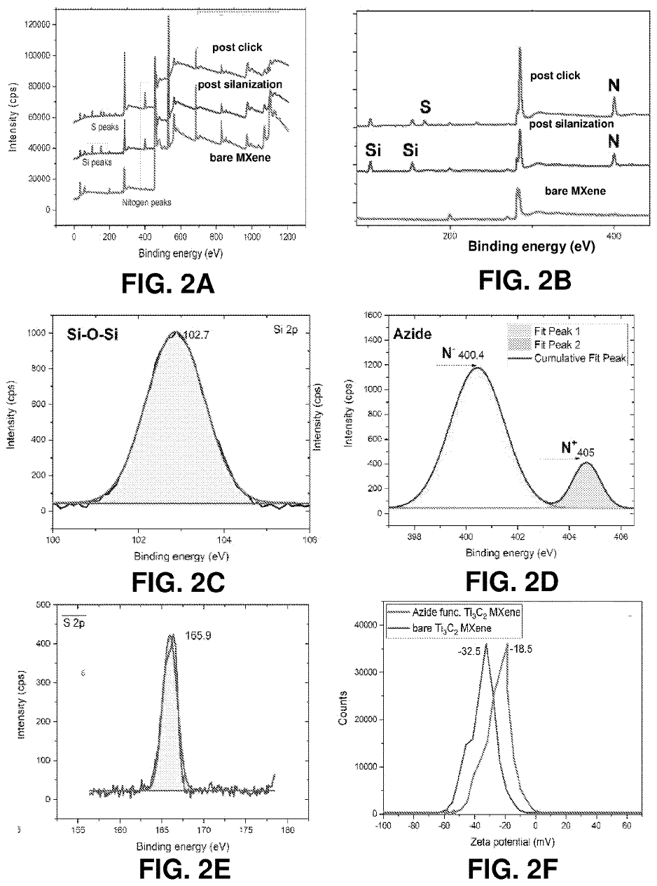

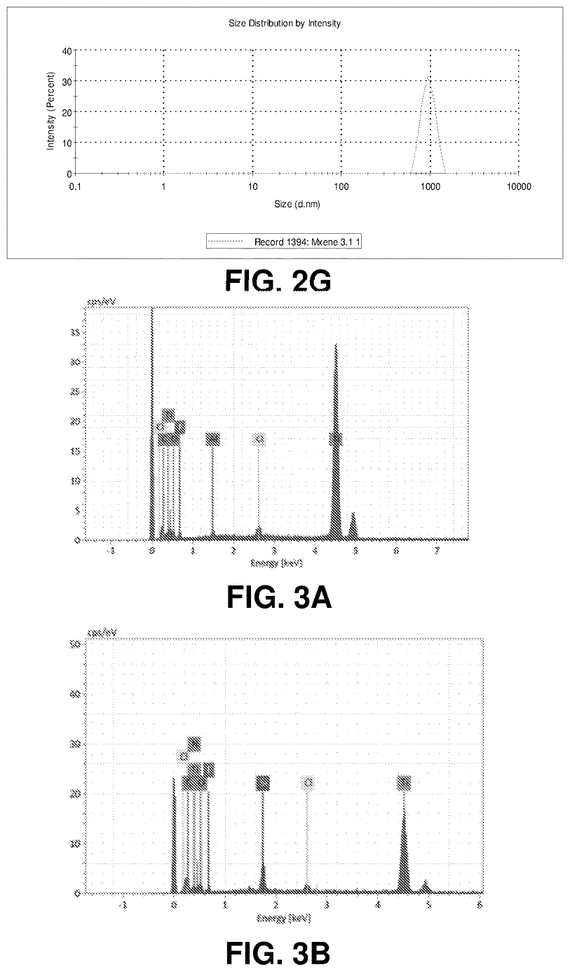

X-ray photoelectron spectroscopy (XPS) was used to analyse the surface chemical states of Ti₃C₂-MXene and functionalized Ti₃C₂-MXene. As shown in FIG. 2A, the XPS spectrum of Ti₃C₂-MXene demonstrates the presence of Ti, C, O and F atoms. The presence of O and F indicates the surface functional groups [Ti₃C₂(OH)₂, Ti₃C₂F₂] appeared following etching.

The presence of Si 2p and N is peaks in functionalized-Ti₃C₂-MXene confirms successful AzPTES functionalization as shown in FIG. 2A. The highlighted peaks are shown in FIG. 2B. Further, the C is peaks at 281.7 eV and 285.7 correspond to internal C—Ti bonds and surface terminated C—Ti bonds (C—Ti—O/F) of the functionalized-Ti₃C₂-MXene, respectively. The peaks at high binding energy (685.1 eV) correspond to the C—F bond. After MXene functionalization, an intense peak for C—N at ~400 eV appeared, indicating the presence of Azide (—N3) functional group on the Ti₃C₂-MXene nanosheets (FIG. 2B). Additionally, the peak at 532.2 eV corresponds to oxygen bonded to surface titanium; the peak at 532.8 eV is assigned to C—Ti—OH moiety (FIG. 2A). After functionalization, Ti—OH moiety forms a covalent bond with the silicon atom in the AzPTES. This is shown in FIG. 2C, where an intense Si—O peak at 532.4 eV is observed. To confirm the presence of Azide groups on the surface of the AzPTES-Ti₃C₂Tx, N is high resolution XPS spectrum was acquired (FIG. 2D). As shown in FIG. 2D, the N is spectrum is deconvoluted into two peaks corresponding to N− species and N+ species at binding energies of 400.2 and 405 eV, respectively. The spectrum demonstrates the presence of the C—N═N═N on the MXene surface.

To validate successful click chemistry, a “clickable” DBCO-modified dye was used as an indicator for the DBCO-PNA probe, as the dye contains element sulphur which is not present in other materials forming the PNA functionalized-Ti₃C₂-MXene. BP Fluor 647 DBCO was used to react with azides to produce a stable triazole bond without requiring a copper catalyst or high temperatures. Successful Click reaction was confirmed by the appearance of the S peak on the XPS spectrum as shown in FIG. 2E, as well as an increase in the percentage of nitrogen peak due to the triazole formation.

The zeta-potential of the nanosheets was also investigated using Zetasizer to study the stability and surface charge of the pure and modified MXene nanosheets (FIG. 2F). As shown in FIG. 2F, the pure MXene in water had a surface potential of −32.5 mV, indicating a stable dispersion colloidal solution. After azide surface functionalization, the zeta potential of the f-MXene nanosheets changed to −18.5 mV, indicating the reduction of OH⁻ groups, which have reacted during the silanization step. Additionally, DLS of the bare MXene was carried out as shown in FIG. 2G.

Scanning electron microscope (SEM, Zeiss Merlin Compact, Germany) with energy-dispersive X-ray (EDX) was used to investigate the morphologies and perform elemental analysis of pure Ti₃C₂Tₓ-MXene (data not shown). The SEM images of azido-functionalized Ti₃C₂Tₓ and PNA-clicked-MXene were also collected (data not shown). Pure Ti₃C₂Tₓ shows a multilayer accordion-like structure as shown in the SEM micrograph. After the AzPTES modification, the Azide-Ti₃C₂Tx nanosheets exhibit closely layered structures, where the sheets are more confined (less separated). The surface is confined with a coating that has crumbled and a tissue-like structure. A similar morphology can be observed in the SEM images after PNA modification on the MXene nanosheets.

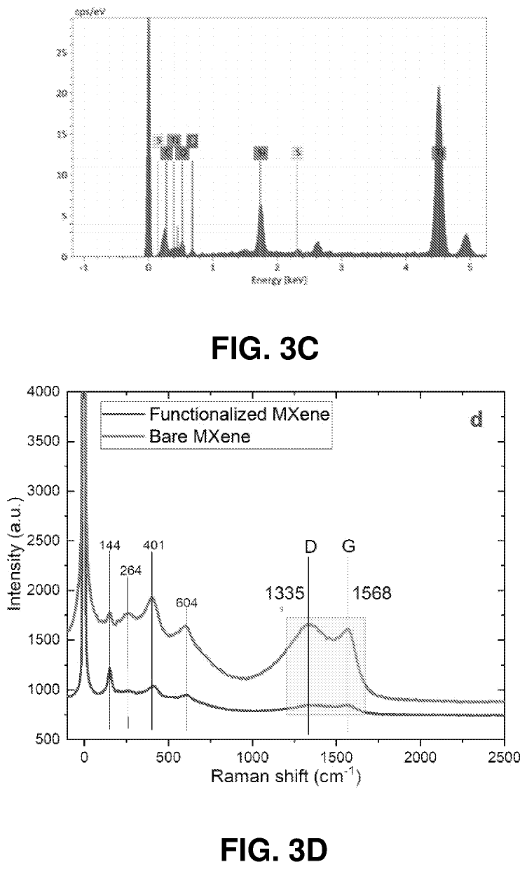

Energy dispersive X-ray spectroscopy (EDX) analysis of pure Ti₃C₂ MXene, azide functionalized Ti₃C₂ MXene, and PNA-MXene is shown in Table 2. The EDX result of Azido-modified MXene nanosheets reveals the presence of Si and N elements, which are distributed uniformly on the MXene nanosheets (6.78% Si, data not shown). Furthermore, the presence of the additional element S uniformly on PNA-MXene (post-Click reaction) confirms successful Click chemistry between the azide on MXene and the proxy DBCO-dye of PNA (6.8% Si, 1.3% S; data not shown). Using EDX (20 keV) coupled with an EMAX energy spectroscope, the surface elements were identified (FIGS. 3A-3C). The elemental composition of Ti₃C₂Tx (FIG. 3A), post silanization (FIG. 3B), and post click reaction (FIG. 3C) were determined. This further demonstrates the formation of Ti₃C₂Tₓ MXene sheets with layers containing azide and triazole functional groups (following Click reaction).

TABLE 2
EDX analysis results

| Sample | Ti | O | C | F | Si | S |
|---|---|---|---|---|---|---|
| Pure Ti₃C₂Tx | 44.2 | 9.59 | 13.2 | 6.69 | | |
| Post silanization Ti₃C₂Tx | 36.40 | 16.93 | 15.41 | 6.25 | 6.78 | |
| Post click reaction Ti₃C₂Tx | 40.15 | 14.98 | 12.25 | 4.86 | 6.81 | 0.38 |

The Raman spectra of pure Ti₃C₂ MXene and azide functionalized Ti₃C₂ MXene are shown in FIG. 3D. The pure (unmodified) Ti₃C₂ MXene exhibited Raman peaks at 264 and 401 cm⁻¹, corresponding to A Ig symmetry out-of-plane vibrations of Ti and C atoms respectively, and at 604 cm⁻¹ corresponding to the Eg group vibrations, including in-plane (shear) modes of Ti, C, and surface functional group atoms. More prominently, there is a peak at roughly 144 cm⁻¹; the high intensity of this peak could be due to the increased laser power, leading to the formation of oxidized Ti₃C₂. Two Raman scattering peaks at 1335 cm⁻¹ (corresponding to the D band) and 1568 cm⁻¹ (corresponding to the G band) can be observed on the spectrum, indicating the presence of graphite carbon. The increase in I_{D}/I_{G} ratio demonstrates the defects in the MXene nanosheets caused by the silane grafting, which may lead to changes in the physical, chemical, structural, and electrical properties of the MXene. The Raman Spectra characterization illustrates that the functionalization of MXene with AzPTES altered the structure of MXene through the formation of more sp³-hybridized carbons within the generally sp²-hybridized carbon network, resulting in higher I_{D}/IG in the azide-functionalized MXene.

### Electrochemical Characterizations

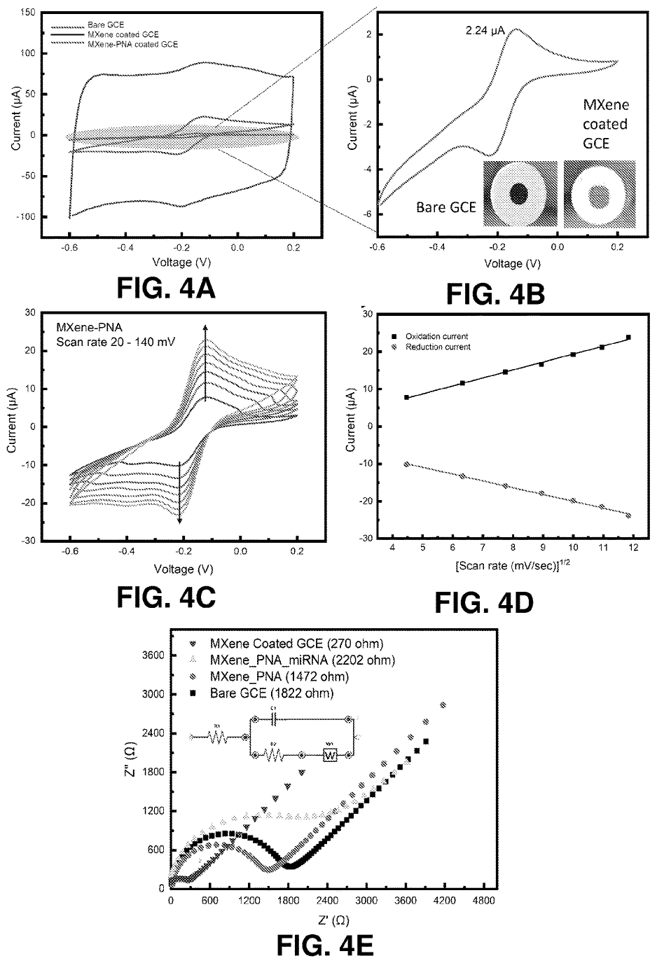

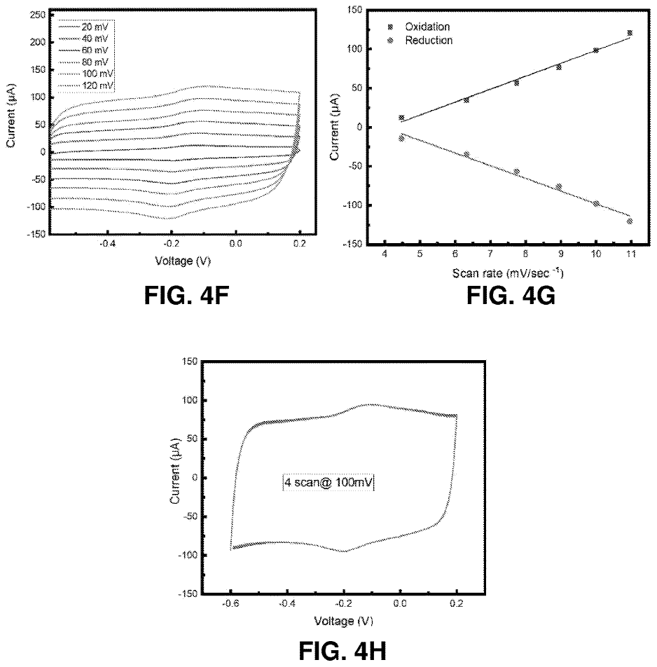

The electrochemical analysis of PNA-Ti₃C₂-GCE was conducted using cyclic voltammetry to investigate the electrochemical process at the electrode/solution interface. Measurements were carried out on the corresponding electrodes in the presence of 5 mM [Ru (NH₃)₆] Cl₃ with voltage sweeping across the potential range from −0.6 V to 0.2 V at a scan rate of 100 mVs⁻¹. Hexaammineruthenium (III) chloride (5 mM) [(Ru (NH₃)₆]Cl₃ was used as a redox probe as it can be oxidized and reduced at lower potential windows (more cathodic) to avoid the oxidation of MXene. The electrodes exhibited differences in the current response values and distinct peak-to-peak separations (ΔEp=Epa−Epc) before- and post-functionalization (FIGS. 4A and 4B). Two well-defined reversible redox peaks were observed at the bare GCE, which correspond to the oxidation and reduction of [Ru (NH₃)₆]Cl₃ (FIG. 4B). When the GCE is modified with f-Ti₃C₂ MXene, the oxidation peak current of the redox probe increased from 1.6 ρA to 86 ρA. This demonstrates the increased speed of electron transfer in the f-MXene nanosheets, which is attributed to the high surface area and electronic conductivity of the MXenes nanosheets. Cyclic voltammetry is used to calculate the active surface areas of the MXene-coated and bare GCE. According to the Randles-Sevcik equation, the electrochemical active area is calculated as follows: Ip=2.69×10⁵AD^{1/2}cv^{1/2}, where Iₚ is the peak current (in amp), A is the electrochemical active area (in cm²), D is the diffusion coefficient of (6.0×10⁻⁶ cm²/s) redox probe used, c is the concentration of [Ru(NH₃)₆]³⁺ (5 mM), and v is the scan rate (in V/s). Calculated using this formula, the active surface area of bare GCE is 0.00543 cm², and the active surface area of MXene coated GCE is 0.0292 cm². Thus, MXene has increased the electrochemically active area of the working electrode by 58.4-fold. This significant increase in active surface area allows high loading of the PNA, which eventually increases the sensing performance, such as the sensitivity, of the E-biosensor.

The PNA loading was determined to be 1.02 moles (based on the amount of the MXene). After PNA immobilization on the MXene nanosheets, a significant drop in the current was observed (data not shown), can be attributed to the high loading of the neutral PNA probes throughout the nanosheet that ultimately impede the permeability of redox probe [Ru (NH₃)₆]³⁺.

The electrochemical kinetics of the f-Ti₃C₂-MXene/GC electrode were studied at scan rates ranging from 20 to 140 mVs⁻¹ (FIG. 4F). The cathodic and anodic current increased progressively with the square root of the scan rate (FIG. 4G), indicating that the redox reaction is reversible and diffusion controlled. Further, a run of multiple scans was carried out to investigate the stability of the electrode (FIG. 4H). Similarly, the electrochemical kinetics of the PNA-Ti₃C₂-MXene-GCE were studied at scan rates ranging from 20 to 140 mVs⁻¹ (FIG. 4C). The cathodic and anodic current increased linearly with the square root of the scan rate as shown in FIG. 4D. This indicates that the PNA probes do not affect the electrochemical behaviour of the redox species generated at the electrode surface, so the mechanism is still diffusion-controlled and electron transfer is reversible.

Additionally, EIS was used to characterize the various GCE modifications. As shown in FIG. 4E, the unmodified GCE provided a small semicircle with a large electron-transfer resistance (Rₑₜ) of 1.82 kΩ. When Ti₃C₂ MXene is deposited on the surface of the GCE, the impedance is reduced by over 5-fold to 270Ω. This demonstrates that the modification of the GCE with the 2D nanomaterial created robust high-performance electron transfer and inferred high conductivity. Modifying the GCE with the PNA-functionalized MXene, however, resulted in an increase in the impedance to 1.47 kΩ, demonstrating high loading of the non-conductive PNA probes throughout the MXene nanosheet. The impedance was further increased to 2.2 kΩ after miRNA hybridization, indicating the reduced charge transfer at the electrode surface in the presence of PNA-miRNA duplex. Consistent results were obtained in terms of charge transfer resistance (Rₑₜ) based on both electrochemical characterization approaches, which further validates the results.

### Biosensor Sensitivity

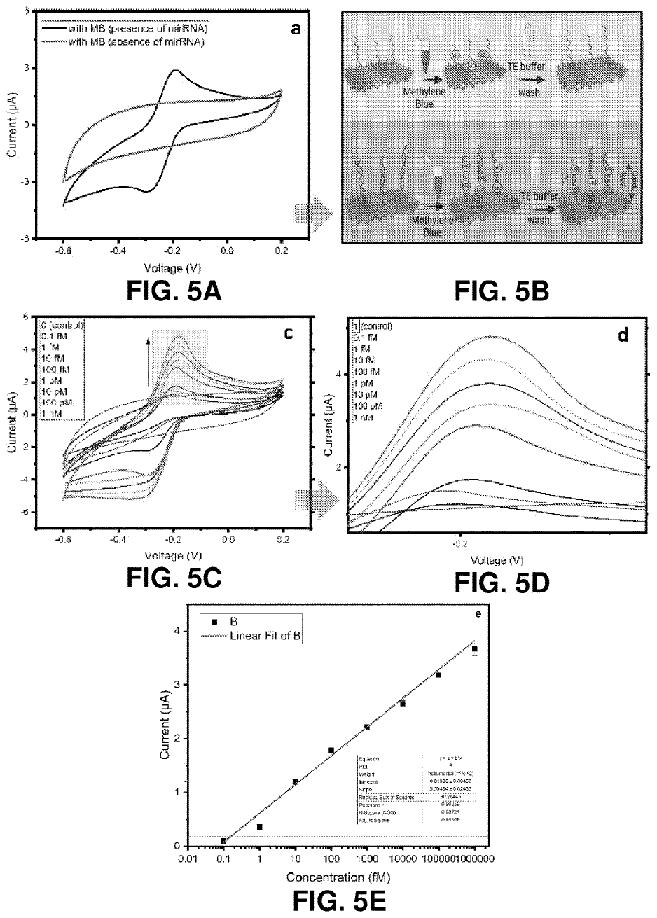

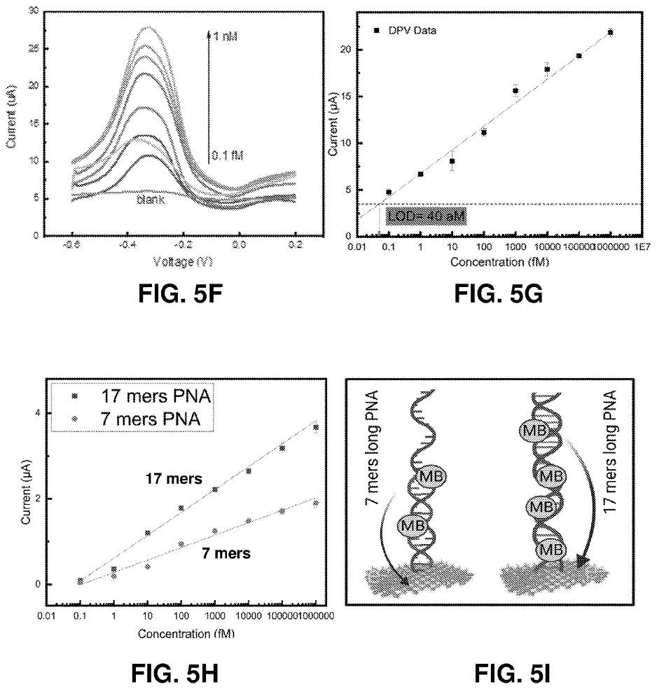

The oxidation peak was obtained by recording the cyclic voltammogram of f-Ti₃C₂ MXene-GCE in the potential range from −0.6 to 0.2 V at a scan rate of 0.1 Vs⁻¹ in the presence of Methylene blue (FIG. 5B, top panel). When no target miRNA was present, and after MB addition, no distinct peak was observed in the CV as shown in FIG. 5A (see also FIG. 5B, top panel). Only capture of the target miRNA by the sequence-specific PNA resulted in a redox reaction due to the presence of the redox probe (MB), which is bound to the miRNA strand through one or a combination of these interactions: intercalation, electrostatic interactions, and interaction with guanine nucleobase. Presence of the MB at the electrode surface (as a result of target capture) results in an oxidation peak at −0.19 V (black curve, FIG. 5A and FIG. 5B, bottom panel). As no other component is redox active, this peak demonstrates the presence of MB at the electrode surface mediated by the capture of negatively charged miRNA through the neutral PNA probes on the Ti₃C₂ MXene-PNA GCE surface. Two different sequences of PNA, i.e., 7 and 17 mers, were investigated. The target miRNA was first captured using a short sequence of 7 mers PNA and based on the interaction of MB after hybridization, a calibration curve was then plotted (FIG. 5H). Then, miRNA-141, which had displayed a strong signal, was captured using a 17 mers PNA utilized as a detection probe (FIG. 5I). The fundamental benefit of a long PNA sequence is that it can carry more MB molecules, increasing sensitivity as shown in FIGS. 5H and 5I. It is hypothesized the MB engages the hybridized complex by electrostatic, intercalation, and groove binding. In this case, the signal quality improved because of the groove binding. Eventually, 17 mers PNA for sensor fabrication was used.

To quantitatively investigate the biosensor analytical performance and sensitivity, a calibration curve was established (FIG. 5E) by monitoring the oxidation peak current as a function of target miR-141 concentration. miRNA of various concentrations was added onto the electrode to allow hybridization with PNA probe for 30 min at RT. The CV curves for the various concentrations are shown in FIG. 5C. After three washes with TE buffer, 5 μL of MB was incubated with the electrode for 5 min at RT, then the electrode was washed thoroughly before recording the CV. FIG. 5C shows the CVs in the presence of target miRNA concentrations ranging from 1 fM to 1 nM. FIG. 5D shows the oxidation peaks at different concentrations. The calibration curve between the electrochemical current and miRNA-141 concentration is plotted, as shown in FIG. 5E. The electrochemical current has a linear relationship with the logarithmic miRNA-141 concentration ranging from 0.1 fM to 1 nM with a LOD of 180 fM, [LOD=3(Std. control)]. The experiment was repeated three times independently using three different sensors and results plotted in the figure above. A high reproducibility and high analytical performance was observed under Biosensor (data not shown). Since DPV measurements (FIG. 5F) can provide higher sensitivity than CV measurements (due to the low capacitive current), a calibration curve was also conducted using this method (FIG. 5G). 5 μL of different concentrations of the miRNA solutions were introduced to the electrode surface and consequently incubated at RT for 30 min prior to recording the DPV measurements (FIG. 5F). A calibration curve of peak oxidation current versus miRNA concentration from 0.1 fM to nM is shown in FIG. 5G.

### Investigating Sequence-Specificity of the Platform

To study the sequence specificity of the sensor, the electrochemical responses of the biosensor in the presence of the target miRNA sequence (miR-141) in comparison to a non-target miRNA (miR-39) in addition to three mutated miR-141 sequences designed with a different number of point mutations were investigated. In particular, the response to either one, two or three site mutations along the miRNA sequence were tested. All sequences tested are shown in Table 1. As shown in FIG. 6, the biosensor showed a significantly lower current response with non-target miRNA, even mutated sequences with as low as a single nucleotide mutation. The signal from the target biomarker (miR-141, green) was normalized to 100%. Next four non-target biomarkers, with sequences described in Table 1, were investigated: the non-target cel-miR-39, and miR-141 with a single mutation, two mutations, or three nucleotide mutations.

All non-target biomarkers generated current signals significantly lower than the target (unpaired, one-tailed t-test). Remarkably, even the microRNA with only a single mutation difference from the target (i.e. miR-141-1SNP) generated below 30% signal, indicating the extremely high sequence specificity of the sensor exemplified herein using PNA probes as selective bioreceptors. It is noteworthy that as the number of mutations increase, the signal generated by the sensor decreases and reaches zero levels with only three mutations within the 22-nucleotide sequence.

All the statistical tests were performed by using Origin software. The results were analyzed through conducting an unpaired, one-tailed t-test (two groups) without assuming equal variance (Welch corrections), wherein p<0.05 was considered statistically significant. In the Figures, *** indicates a p-value below 0.0001.

The high specificity of the sensor may be attributed to the synthetic peptide nucleic acid probes, which is exploited herein as opposed to the conventionally and commonly used ssDNA probes, commonly used in such electrochemical nucleic acid sensors. Due to PNA's neutral backbone, mutations in target nucleic acid sequence cause a greater destabilizing effect when using a PNA probe compared to a ssDNA or RNA probe. These results demonstrated the immense potential of the sensor in nucleic acid detection, especially when applied to microRNA biomarkers which suffer from high sequence homology among family members.

The same biofunctionalization strategy (silanization and click) can be used to functionalize another bioreceptor like an aptamer (ssDNA), a peptide, an antibody, or an antibody fragment.

### Evaluation of the Biosensor Stability and Performance

Developing robust point-of-care biosensors necessitates thorough investigation of their reproducibility and stability over time. To this end, four sensors were prepared independently, and their current responses in the presence of 10 mM RuHex probe was examined by DPV analysis. Sensors 1 and 2 were evaluated immediately after fabrication (also referred to herein as “fresh Sensors”), while Sensors 3 and 4 were tested after seven days of storage at 4° C. As shown in FIG. 7, sensors prepared and stored under identical conditions exhibited remarkable reproducibility. Further, seven days of storage only caused a minimal (specifically, 11%) decrease in their performance.

### CONCLUSION

An exemplary enzyme-free and nanoparticle-free MXene-based electrochemical biosensor for microRNA detection was demonstrated, which was the first example of a PNA-MXene hybrid for electrochemical sensing. The outstanding analytical performance of this enzyme-free platform can be attributed to the highly active hybrid transducer surface, which offers high bioreceptor loading and rapid target biomarker access. This may be achieved by the distinct morphology and surface functional groups of the 2D ultrathin MXene nanosheets and the highly efficient and robust Click chemistry used for biofunctionalization to attach biorecognition elements on the surface of MXene. The example demonstrates the use of engineered MXene-PNA hybrids to advance the field of electrochemical sensing towards more robust, simple, and cost-effective strategies amenable for point-of-care.

### REFERENCES

[1] D. P. Bartel, Cell 136(2) (2009) 215-233.
[2] T. Ochiya and R.-u. Takahashi, MicroRNAs: Novel Biomarkers and Therapeutic Targets for Human Cancers, MDPI2018.
[3] J. Wang, et al., Journal of cellular physiology 231(1) (2016) 25-30.
[4] C. N. Correia, et al., Frontiers in immunology 8 (2017) 118.
[5] H. Dong, et al., Chem Rev 113(8) (2013) 6207-33.
[6] C. De la Rosa and J. L. Reyes, Plant MicroRNAs, Springer 2019, pp. 121-129.
[7] L. Wu, et al., Biosensors and Bioelectronics 107 (2018) 69-75.
[8] J. Zheng, et al., Journal of the Electrochemical Society 165(5) (2018) B227.
[9] P. A. Rasheed, et al., Sensors and Actuators B: Chemical 265 (2018) 652-659.
[10] X. Cai, et al., Chemical Society Reviews 47(16) (2018) 6224-6266.
[11] M. Naguib, et al., Advanced Materials 23(37) (2011) 4207-4207.
[12] B. Akuzum, et al., ACS nano 12(3) (2018) 2685-2694.
[13] G. Gao, et al., Acs Catalysis 7(1) (2017) 494-500.
[14] R. A. Soomro, et al., Chinese Chemical Letters 31(4) (2020) 922-930.
[15] Q. Wu, et al., Biosensors and Bioelectronics 144 (2019) 111697.
[16] P. Yadav, et al., ACS nano 15(3) (2021) 4861-4869.
[17] X. Yang, et al., Journal of Electroanalytical Chemistry 878 (2020) 114669.
[18] M. Mohammadniaei, et al., Biosensors and Bioelectronics 159 (2020) 112208.
[19] F. Duan, et al., Sensors and Actuators B: Chemical 310 (2020) 127844.
[20] L. Liu, et al., Biosensors and Bioelectronics 137 (2019) 45-51.
[21] F. Wang, et al., Biosensors and Bioelectronics 74 (2015) 1022-1028.
[22] J. Hu, et al., Biotechnology and Bioengineering 116(10) (2019) 2764-2777.
[23] S. Ruffin, et al., ACS sensors 2(7) (2017) 892-896.
[24] M. Naguib, et al., Advanced materials 23(37) (2011) 4248-4253.
[25] J. Halim, et al., Advanced Functional Materials 26(18) (2016) 3118-3127.
[26] A. Gouget-Laemmel, et al., The Journal of Physical Chemistry C 117(1) (2013) 368-375.
[27] C. Sun, et al., Nano-micro letters 13(1) (2021) 1-13.
[28] A. Iqbal, et al., Materials 14(21) (2021) 6292.
[29] F. Liu, et al., Adsorption 22(7) (2016) 915-922.
[30] P. Bandyopadhyay, et al., Journal of Membrane Science 500 (2016) 106-114.
[31] S. Kumar, et al., Biosensors and Bioelectronics 121 (2018) 243-249.
[32] T. R. Paixio, ChemElectroChem 7(16) (2020) 3414-3415.
[33] N. Rohaizad, et al., Chemical Society Reviews 50(1) (2021) 619-657.
[34] H.-A. Rafiee-Pour, et al., Biosensors and bioelectronics 77 (2016) 202-207.
[35] P. Paul, et al., Journal of Photochemistry and Photobiology B: Biology 204 (2020)
[36] B. J. Venton and D. J. DiScenza, Journal: Electrochemistry for Bioanalysis, 2020, p. 27-50.
[37] P. E. Nielsen, Molecular biotechnology 26(3) (2004) 233-248.

1. A hybrid material comprising a plurality of MXene sheets having biorecognition elements conjugated thereto, wherein the biorecognition elements comprise a binding partner to an analyte of interest.

2. The hybrid material of claim 1, wherein the biorecognition elements are conjugated to one or more surfaces of the MXene sheets via a linker, and wherein the linker is represented by the formula: 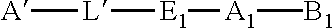
wherein A′ is 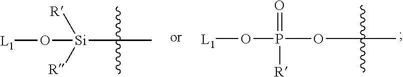
L₁ is an attachment point to the surface of MXene;
R′ and R″ are independently 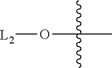
or a C₁-C₄ unsubstituted alkyl;
L₂ is an attachment point to the surface of MXene, a C₁-C₄ unsubstituted alkyl, or hydrogen;
L′ is 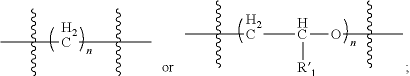
each occurrence of n is independently an integer from 1 to 20;
R′₁ is hydrogen or C₁-C₄ unsubstituted alkyl (e.g., methyl, ethyl, etc.);
E₁ is a bond, a peptide, or 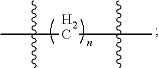
A₁ is a moiety formed by click chemistry; and
B₁ is the biorecognition element,
Optionally wherein the linker is represented by the formula: 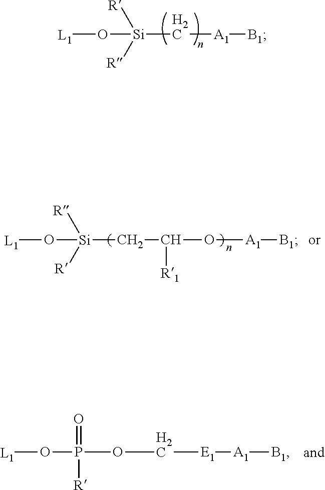
optionally wherein A₁ is

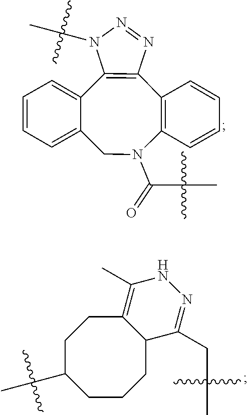 [SMILES: *C(=O)N1Cc2ccccc2-c2c(nnn2*)-c2ccccc21 ; *CC1=NNC(C)=C2CCC(*)CCCC12]

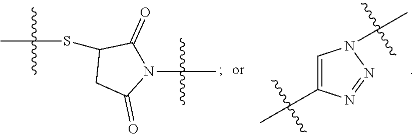 [SMILES: *SC1CC(=O)N(*)C1=O ; *c1cn(*)nn1]

3. The hybrid material of claim 2, wherein one of R′ and R″ is 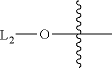
and L₂ is an attachment point to the one or more surfaces of the MXene sheets, or wherein both R′ and R″ are 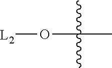
and L₂ is an attachment point to the one or more surfaces of the MXene sheets.

4. The hybrid material of claim 1, wherein: (a) the biorecognition element is a nucleic acid, a peptide, or a protein (e.g., an antibody, a nanobody, an antibody fragment, or an enzyme), optionally wherein the biorecognition element is a peptide nucleic acid and/or (b) the analyte of interest is a biomarker,
optionally wherein the analyte of interest is a nuclei acid, a protein, a pathogen, a component of a pathogen, a hormone, a carbohydrate, a cell, an extracellular vesicle, or a virus, optionally wherein the analyte of interest is a miRNA.

5. (canceled)

6. The hybrid material of claim 1, wherein each MXene sheet comprises
a layered body represented by Mₙ₊₁Xₙ, wherein n is an integer from 1 to 4, each M is a metal of Group 3, 4, 5, 6, or 7, and each X is a carbon atom or a nitrogen atom; and
optionally one or more surface functional groups.

7. The hybrid material of claim 6, wherein each M is selected from the group consisting of Ti, Zr, Hf, V, Nb, Ta, Cr, and Mo; and/or wherein the functional group(s) is/are hydroxyl, oxide, halogen, or carbonyl, or a combination thereof.

8. (canceled)

9. The hybrid material of claim 1, wherein the MXene sheets are Ti₃C₂ nanosheets and optionally with one or more surface hydroxyl groups.

10. A sensor comprising a working electrode,
wherein the working electrode comprises a substrate and the hybrid material of claim 1 deposited thereon.

11. The sensor of claim 10, wherein the hybrid material forms a coating on one or more surfaces of the substrate, optionally wherein the coating is a porous coating; and/or wherein the substrate is formed from a conductive material, optionally wherein the conductive material is carbon-based material, a metal, or a metal alloy, optionally wherein the substrate is formed from glassy carbon or gold.

12. (canceled)

13. The sensor of claim 10, wherein the substrate is formed from a non-conductive film.

14. The sensor of claim 10, further comprising a counter electrode and optionally a reference electrode, optionally wherein the counter electrode is platinum, optionally wherein the reference electrode is Ag/AgCl.

15. A method of making the hybrid material of any one of claims 1-9, comprising:
(a) treating MXene sheets having a plurality of surface hydroxyl groups with a clickable group functionalized silane or phosphonic acid having the structure of 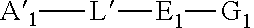
wherein A′₁ is 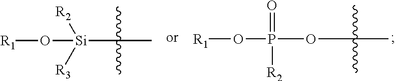
R₁ is a C₁-C₄ unsubstituted alkyl;
R₂ and R₃ are independently 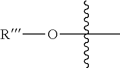
a C₁-C₄ unsubstituted alkyl;
R′″ is hydrogen or a C₁-C₄ unsubstituted alkyl;
L′ is 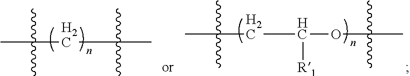
each occurrence of n is independently an integer from 1 to 20, such as from 1 to 6;
R′₁ is hydrogen or C₁-C₄ unsubstituted alkyl (e.g., methyl, ethyl, etc.);
E₁ is a bond, a peptide, or 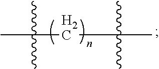
and
G₁ is a clickable group,
to form clickable group functionalized MXene sheets, and
(b) reacting the clickable group functionalized MXene sheets with a clickable group functionalized biorecognition element G₂-B₁, wherein B₁ is the biorecognition element and G₂ is a corresponding clickable group capable of reacting with the clickable group of the clickable group functionalized MXene sheets via Click Chemistry,
to form the hybrid material,
optionally wherein the clickable group functionalized silane or phosphonic acid has the structure of 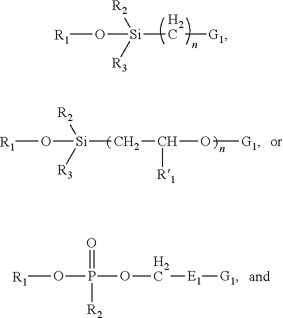
optionally wherein G₁ and G₂ are selected from the group consisting of

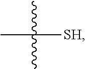 [SMILES: *S]

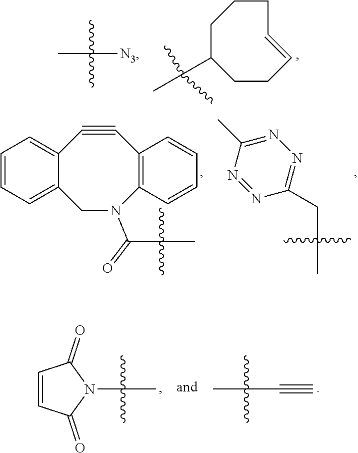

16. The method of claim 15, wherein: (a) in step (a), the mole amount of clickable group functionalized silane or phosphonic acid is in excess to the mole amount of surface hydroxyl groups, (b) step (a) is performed in a solvent, such as ethanol, or without a solvent; and/or (c) step (a) is performed at room temperature (20° C.-25° C. at 1 atm) for a time period ranging from about 30 mins to about 60 hours.

17. (canceled)

18. (canceled)

19. The method of claim 15, (a) further comprising washing and centrifugating the clickable group functionalized MXene sheets, after step (a) and prior to step (b), to remove unreacted clickable group functionalized silane or phosphonic acid; (b) wherein in step (b), the weight ratio of clickable group functionalized MXene sheets to clickable group functionalized biorecognition element is in a range from 10:1 to 1:10 or from 1:5 to 1:2; (c) wherein step (b) is performed in a solvent, such as water; and/or (d) wherein step (b) is performed at room temperature for a time period ranging from about 30 mins to about 18 hours.

20. (canceled)

21. (canceled)

22. (canceled)

23. The method of claim 15, further comprising washing the hybrid material after step (b) to remove unreacted clickable group functionalized biorecognition element; and/or wherein step (a) and/or (b) is performed under agitation, such as stirring.

24. (canceled)

25. A method of detecting the absence, the presence, and/or the concentration of an analyte of interest in a liquid biological sample, comprising
(i) contacting the working electrode of the sensor of any one of claims 10-14 with the biological sample;
(ii) optionally contacting the working electrode of the sensor with a redox reporter solution; and
(iii) measuring an electrochemical signal generated by the sensor.

26. The method of claim 25, (a) further comprising (iv) contacting the working electrode of the sensor with the redox reporter solution; and (v) measuring a background signal generated by the sensor, wherein steps (iv) and (v) are performed prior to step (i); (b) wherein the redox reporter is methylene blue, ferrocene, ferrocyanide, ferricyanide, tris(bipyridine)ruthenium(II) chloride, hexaammineruthenium(III) chloride, or anthraquinone, or a combination thereof; (c) wherein in step (iii), the electrochemical signal is measured using a method selected from the group consisting of cyclic voltammetry, differential pulse voltammetry, square wave voltammetry, amperometry, or electrochemical impedance spectroscopy, or a combination thereof; (d) wherein in step (v), the background signal is measured using the same method as the electrochemical signal measured in step (iii); (e) wherein each of step (iii) and step (v) is preformed in a buffer solution, optionally wherein the buffer solution is a TE buffer at physiological pH; and/or (f) wherein in steps (i), the working electrode is in contact with the biological sample for a time period ranging from about 1 min to about 4 hours.

27. (canceled)

28. (canceled)

29. (canceled)

30. (canceled)

31. (canceled)

32. The method of claim 25, wherein in each of step (ii) and step (iv), the working electrode is in contact with the redox reporter solution for a time period ranging from about 1 min to about 10 mins or from about 1 min to about 5 mins, at room temperature.

33. The method of claim 25, further comprising washing the working electrode one or more times after step (i) and prior to step (ii), after step (ii) and prior to step (iii), and/or after step (iv) and prior to step (v) and/gr wherein the biological sample is (a) a bodily fluid, (b) an extract of a bodily fluid, or (c) a non-bodily fluid (e.g., a buffer or an environmental fluid), or a combination thereof, optionally wherein the bodily fluid is selected from the group consisting of whole blood, plasma, serum, saliva, interstitial fluid, nasal fluid, tears, breast milk, mucus, sputum, bronchial alveolar lavage (BAL), bronchial wash (BW), cerebrospinal fluid (CSF), and urine.

34. (canceled)

35. The method of claim 25, wherein: (a) the volume of the biological sample is ≤20 μL, ≤10 μL, or ≤5 μL; (b) the sensor has a limit of detection down to about 40 aM, such as about 180 fM, of the analyte of interest; (c) the sensor has a detection dynamic range from about 40 aM to about 1 nM, such as from about 0.1 fM to about 1; and/or (d) wherein the sensor has a shelf stability of at least 1 week, at least 1 month, at least 3 months, or at least 6 months, at 4° C.

36. (canceled)

37. (canceled)

38. (canceled)
